(19) United States
(12) Patent Application Publication
(10) Pub. No.: US 2017/0119776 A1
(43) Pub. Date: May 4, 2017

(54) CHIRAL 2,5-DISUBSTITUTED CYCLOPENTANECARBOXYLIC ACID DERIVATIVES AND USE THEREOF

(71) Applicant: BAYER PHARMA AKTIENGESELLSCHAFT, Berlin (DE)

(72) Inventors: Hartmut BECK, Wuppertal (DE); Volkhart Min-Jian LI, Velbert (DE); Andreas TIMMERMANN, Düsseldorf (DE); Pamela BOGNER, München (DE); Yolanda CANCHO GRANDE, Leverkusen (DE); Dirk BROHM, Mettmann (DE); Michael GERISCH, Wuppertal (DE); Hannah JÖRISSEN, Heiligenhaus (DE); Dieter LANG, Velbert (DE)

(21) Appl. No.: 15/300,436

(22) PCT Filed: Mar. 31, 2015
(86) PCT No.: PCT/EP2015/056979

§ 371 Date: Sep. 29, 2016

(30) Foreign Application Priority Data
Apr. 3, 2014 (EP) 14163308.1

(51) Int. Cl. A61K 31/53; A61K 45/06; C07D 253/08

(52) U.S. Cl. CPC A61K 31/53; C07D 253/08; A61K 45/06

(57) ABSTRACT

The present application relates to novel, chiral 2,5-disubstituted cyclopentanecarboxylic acid derivatives, to a method for their preparation, to their use on their own or in combinations for the treatment and/or prevention of diseases, and to their use for producing medicaments for the treatment and/or prevention of diseases, in particular for the treatment and/or prevention of diseases of the respiratory tracts, the lung and the cardiovascular system.

The present application relates to novel, chiral 2,5-disubstituted cyclopentanecarboxylic acid derivatives, to a process for their preparation, to their use on their own or in combinations for the treatment and/or prevention of diseases, and to their use for producing medicaments for the treatment and/or prevention of diseases, in particular for the treatment and/or prevention of diseases of the respiratory tract, the lung and the cardiovascular system.

Human macrophage elastase (HME, EC 3.4.24.65) belongs to the family of matrix-metallo-peptidases (MMPs) and is also called human matrix-metallo-peptidase 12 (hMMP-12). The protein is formed, activated and released to an increased extent inter alia by macrophages following contact with “irritative” substances or particles. Such substances and particles can be present for example as foreign substances in suspended particles, as occur inter alia in cigarette smoke or industrial dusts. In the wider sense, endogenous and exogenous cell constituents and cell debris are also included among these irritative particles, as can be present in sometimes high concentration during inflammatory processes. The highly active enzyme is able to degrade a large number of connective tissue proteins, e.g. primarily the protein elastin (hence the name), and further proteins and proteoglycans such as collagen, fibronectin, laminin, chondroitin sulphate, heparin sulphate and more besides. This proteolytic activity of the enzyme enables the macrophages to penetrate the basal membrane. Elastin for example occurs in high concentrations in all types of tissue which exhibit high elasticity, e.g. in the lung and in arteries. In a large number of pathological processes, such as tissue damage, HME plays an important role in tissue degradation and remodelling. Moreover, HME is an important modulator in inflammatory processes. It is a key molecule in the recruitment of inflammatory cells by, for example, releasing the central inflammation mediator tumour necrosis factor alpha (TNF-α) and intervening in the signal pathway mediated by transforming growth factor-beta (TGF-β) [Hydrolysis of a Broad Spectrum of Extracellular Matrix Proteins by Human Macrophage Elastase, Gronski et al., J. Biol. Chem. 272, 12189-12194 (1997)]. MMP-12 also plays a role in host defence, particularly for the regulation of antiviral immunity, presumably as a result of an intervention into the interferon-alpha (IFN-α)-mediated signal pathway [A new transcriptional role for matrix metalloproteinase-12 in antiviral immunity, Marchant et al., Nature Med. 20, 493-502 (2014)].

It is therefore assumed that HME plays an important role in many diseases, injuries and pathological changes whose aetiology and/or progression is associated with an infectious or noninfectious inflammatory event and/or a proliferative and hypertrophic tissue and vessel remodelling. These can be in particular diseases and/or damage to the lung, the kidney or the cardiovascular system, or they can be cancer diseases or other inflammatory diseases [Macrophage metalloelastase (MMP-12) as a target for inflammatory respiratory diseases, Lagente et al., Expert Opin. Ther. Targets 13, 287-295 (2009); Macrophage Metalloelastase as a major Factor for Glomerular Injury in Anti-Glomerular Basement Membrane Nephritis, Kaneko et al., J. Immunol. 170, 3377-3385 (2003); A Selective Matrix Metalloelastase-12 Inhibitor Retards Atherosclerotic Plaque Development in Apolipoprotein E Knock-out Mice, Johnson et al., Arterioscler. Thromb. Vasc. Biol. 31, 528-535 (2011); Impaired Coronary Collateral Growth in the Metabolic Syndrome Is in Part Mediated by Matrix Metalloelastase 12-dependent Production of Endostatin and Angiostatin, Dodd et al., Arterioscler. Thromb. Vasc. Biol. 33, 1339-1349 (2013); Matrix metalloproteinase pharmacogenomics in non-small-cell lung carcinoma, Chetty et al., Pharmacogenomics 12, 535-546 (2011)].

Diseases and damage to the lung to be mentioned in this context are in particular chronic obstructive pulmonary disease (COPD), pulmonary emphysema, interstitial lung diseases (ILD) such as e.g. ideopathic pulmonary fibrosis (IPF) and pulmonary sarcoidosis, acute lung injury (ALI), acute respiratory distress syndrome (ARDS), cystic fibrosis (CF; also called muscoviscidosis), asthma, as well as infectious, in particular virally induced respiratory tract diseases. Other fibrotic diseases which may be mentioned here by way of example are liver fibrosis and systemic sclerosis. Diseases and damage to the cardiovascular system in which HME is involved are, for example, tissue and vascular changes in arteriosclerosis, here in particular carotid arteriosclerosis, infective endocarditis, here in particular viral myocarditis, cardiomyopathy, heart insufficiency, cardiogenic shock, acute coronary syndrome (ACS), aneurysms, reperfusion injuries following an acute myocardial infarct (AMI), ischaemic injuries to the kidneys or the retina, as well as their chronic courses, such as for example chronic kidney disease (CKD) and Alport's syndrome. Mention may also be made here of metabolic syndrome and obesity. Diseases related to sepsis are, for example, systemic inflammatory response syndrome (SIRS), severe sepsis, septic shock and multiple organ failure (MOF); multiorgan dysfunction (MODS) and also disseminated intravascular coagulation (DIC). Examples of tissue degradation and remodelling during neoplastic processes are the invasion of cancer cells into healthy tissue (formation of metastases) and neovascularization (neoangiogenesis). Other inflammatory diseases in which HME plays a role are rheumatoid diseases, for example rheumatoid arthritis, and also chronic intestinal inflammation (inflammatory bowel disease (IBD); Crohn's disease CD; ulcerative colitis UC).

In general, it is assumed that elastase-mediated pathological processes are based on a shifted equilibrium between the free elastase (HME) and the tissue inhibitor of metalloproteinase (TIMP). In various pathological, in particular inflammatory processes, the concentration of free elastase (HME) is increased, meaning that locally the balance between protease and antiprotease is shifted in favour of the protease. A similar (im)balance exists between the elastase of neutrophil cells (human neutrophil elastase, HNE, a member of the serine protease family) and the endogenous anti-protease AAT (alpha-1 anti-trypsin, a member of the serine protease inhibitors, SERPINs). The two equilibria are coupled together since HME cleaves and deactivates the inhibitor of the HNE, and vice versa HNE cleaves and deactivates the HME inhibitor, as a result of which the respective protease/antiprotease imbalances can additionally shift. Moreover, in the field of local inflammations, strongly oxidizing conditions prevail (oxidative burst), as a result of which the protease/antiprotease imbalance is further intensified [Pathogenic triad in COPD: oxidative stress, protease-antiprotease imbalance, and inflammation, Fischer et al., Int. J. COPD 6, 413-421 (2011)].

Currently, more than 20 MMPs are known, which are historically roughly divided into different classes with regard to their most prominent substrates, e.g. gelatinases (MMP-2, MMP-9), collagenases (MMP-1, MMP-8, MMP-13), stromelysins (MMP-3, MMP-10, MMP-11) and matrilysins (MMP-7, MMP-26). HME (MMP-12) is hitherto the only representative of metalloelastase. Moreover, further MMPs are added to the group of so-called MT-MMPs (membrane-type MMPs) since these have a characteristic domain which anchors the protein in the membrane (MMP-14, MMP-15, MMP-16, MMP-17, MMP-24, MMP-25). A common feature of all the MMPs is a preserved zinc-binding region in the active centre of the enzyme which is important for the catalytic activity and which can also be found in other metalloproteins (e.g. a disintegrin and metalloproteinase, ADAM). The complexed zinc is masked by a sulphhydryl group in the N-terminal pro-peptide domain of the protein, which leads to an enzymatically inactive proform of the enzyme. Only as a result of a cleaving off of this pro-peptide domain is the zinc in the active centre of the enzyme freed from this coordination and the enzyme is thereby activated (so-called activation by cysteine switch) [Matrix metalloproteinase inhibitors as therapy for inflammatory and vascular diseases, Hu et al., Nature Rev. Drug Discov. 6, 480-498 (2007)].

Most of the known synthetic MMP inhibitors provide a zinc-complexing functional group, very often for example a hydroxamate, a carboxylate or a thiol [Recent Developments in the Design of Specific Matrix Metalloproteinase Inhibitors aided by Structural and Computational Studies, B. G. Rao, Curr. Pharm. Des. 11, 295-322 (2005)]. The scaffold of these inhibitors often also resembles peptides, the term used then being so-called peptidomimetics (generally with a poor oral bioavailability), or it has no similarity to peptides, the term used then being more generally small molecules (SMOLs). The physicochemical and pharmacokinetic properties of these inhibitors have, in quite general terms, a major influence on which target molecules (targets) and which undesired molecules (anti-targets, off-targets) are “encountered” in what tissue and in what period to what extent.

It is a major challenge here to determine the specific role of a certain MMP in an incidence of disease. This is made more difficult particularly as a result of the fact that there are a large number of MMPs and further similar molecules (e.g. ADAMs), together with a large number of possible physiological substrates in each case and therefore, under certain circumstances, also associated inhibitory or activatory effects in diverse signal transduction pathways. Numerous in vitro and preclinical in vivo experiments have contributed much to a better understanding of the MMPs in various disease models (e.g. transgenic animals, knock-out animals, as well as genetic data from human studies). The validation of a target as regards a possible medicamentous therapy can ultimately take place only in clinical test series on humans or patients. The first generation of MMP inhibitors in this regard has been clinically investigated in cancer studies. At this time, only a few representatives of the MMP protein family were known. None of the investigated inhibitors were clinically convincing since at effective doses the side effects that arose could not be tolerated. As emerged in the course of the knowledge of further MMPs, the representatives of the first inhibitor generation were non-selective inhibitors, i.e. a large number of different MMPs was inhibited to the same extent (pan-MMP inhibitors, pan-MMPIs). Presumably, the desired effect on one or more MMP targets was concealed by an undesired effect on one or more MMP anti-targets or by means of an undesired effect at another target site (off-target) [Validating matrix metallo proteinases as drug targets and anti-targets for cancer therapy, Overall & Kleifeld, Nature Rev. Cancer 6, 227-239 (2006)].

Newer MMP inhibitors, which are characterized by increased selectivity, have now likewise been clinically tested, including compounds referred to explicitly as MMP-12 inhibitors, although hitherto likewise without compelling clinical success. On looking more carefully, the inhibitors described as being selective beforehand have also turned out to be not quite so selective.

For example, for the clinical test compound “MMP408” as MMP-12 inhibitor, a certain to significant selectivity is described in vitro towards MMP-13, MMP-3, MMP-14, MMP-9, Agg-1, MMP-1, Agg-2, MMP-7 and TACE [A Selective Matrix Metalloprotease 12 Inhibitor for Potential Treatment of Chronic Obstructive Pulmonary Disease (COPD): Discovery of (S)-2-(8-(Methoxycarbonylamino)dibenzo[b,d]furan-3-sulfonamido)-3-methylbutanoic acid (MMP408), Li et al., J. Med. Chem. 52, 1799-1802 (2009)]. In vitro activity data relating to MMP-2 and MMP-8 point to a less advantageous selectivity towards these two MMP representatives [Matrix metalloproteinase-12 is a therapeutic target for asthma in children and young adults, Mukhopadhyay et al., J. Allergy Clin. Immunol. 126, 70-76 (2010)].

The situation is similar with the clinical test substance AZD1236 for the treatment of COPD, which is described as a dual MMP 9/12 inhibitor [Effects of an oral MMP-9 and -12 inhibitor, AZD1236, on biomarkers in moderate/severe COPD: A randomised controlled trial, Dahl et al., Pulm. Pharmacol. Therap. 25, 169-177 (2012)]. The development of this compound was stopped in 2012; here too, a noticeable inhibition of MMP-2 and MMP-13 is cited [http://www.wipo.int/research/en/details.jsp?id=2301].

When assessing the MMP selectivity, moreover, a careful estimation of the meaningfulness of animal models is indicated. For example, the test compound MMP408 shows a significantly reduced affinity to the orthologous MMP-12 target of the mouse: IC₅₀ 2 nM (human MMP-12), IC₅₀ 160 nM (murine MMP-12), IC₅₀ 320 nm (MMP-12 of the rat) [see above Li et al., 2009; Mukhopadhyay et al., 2010]. Data relating to the activity strength towards other MMPs of the mouse are not published. It appears to be a similar case for the test substance AZD1236 [see the information relating to cross-reactivity in various animal species given under http://www.wipo.int/research/en/details.jsp?id=2301].

Besides the selectivity profile beyond species boundaries, the activity strength on the target MMP-12 itself is also very important. For a comparatively similar pharmacokinetic profile, a highly potent compound will lead to a lower therapeutic dose than a less potent compound, and in general a lower dose should be associated with a reduced probability of side effects. This is the case in particular with regard to the so-called “free fraction” (fraction unbound, fᵤ) of a compound which can interact with the desired target and/or undesired anti- and off-targets (the “free fraction” is defined as the available amount of a compound which is not bound to constituents of blood plasma; these are primarily blood protein constituents such as e.g. albumin) Besides the MMP selectivity, the specificity is thus also of prime importance.

New active ingredients inhibiting the macrophage elastase should accordingly have a high selectivity and specificity in order to be able to inhibit the HME in a targeted manner. In this respect, a good metabolic stability of the substances is also necessary (low clearance). Moreover, these compounds should be stable under oxidative conditions in order not to lose inhibitory potency in the disease incidence.

Chronic obstructive pulmonary disease (COPD) is a slowly progressing pulmonary disease characterized by an obstruction of respiratory flow which is caused by pulmonary emphysema and/or chronic bronchitis. The first symptoms of the disease generally manifest themselves during the fourth or fifth decade in life. In subsequent years of life, the shortness of breath often deteriorates, manifesting itself in coughs, associated with extensive and at times purulent sputum and a stenosis respiration ranging to breathlessness (dyspnoea). COPD is primarily a disease of smokers: Smoking is the cause of 90% of all cases of COPD and of 80-90% of all COPD-related deaths. COPD is a big medical problem and constitutes the sixth most frequent cause of death worldwide. Of people over the age of 45, about 4-6% are affected.

Although the obstruction of the respiratory flow may only be partial and temporal, COPD can not be cured. Accordingly, the aim of the treatment is to improve the quality of life, to alleviate the symptoms, to prevent an acute worsening and to slow the progressive impairment of lung function. Existing pharmacotherapies, which have changed little over the last two or three decades, are the use of bronchodilators to open blocked respiratory passages, and in certain situations corticosteroids to control the inflammation of the lung [Chronic Obstructive Pulmonary Disease, P. J. Barnes, N. Engl. J. Med. 343, 269-280 (2000)]. The chronic inflammation of the lung, caused by cigarette smoke or other irritants, is the driving force of the development of the disease. The underlying mechanism involves immune cells which release various chemokines in the course of the inflammatory reaction of the lung. As a result, neutrophil cells and, during further progression, alveolar macrophages are locked to the lung connective tissue and lumen. Neutrophil cells secrete a protease cocktail which primarily contains HNE and proteinase 3. Activated macrophages release the HME. As a result, the protease/antiprotease balance is shifted locally in favour of the proteases, which inter alia leads to an uncontrolled elastase activity and, as a consequence of this, leads to an excessive degradation of the alveolar elastin. This tissue degradation causes a collapse of the bronchi. This is associated with a reduced elasticity of the lung, which leads to a hindering of breath flow and impaired breathing. Moreover, frequent and long-term inflammation of the lungs can lead to a remodelling of the bronchi and consequently to a formation of lesions. Such lesions can contribute to the occurrence of a chronic cough, which characterizes chronic bronchitis.

It is known from experiments with human sputum samples that the amount of HME protein is associated with the smoke or COPD status: The detectable amounts of HME are the lowest in non-smokers and somewhat increased for former smokers and smokers, and significantly increased in COPD patients [Elevated MMP-12 protein levels in induced sputum from patients with COPD, Demedts et al., Thorax 61, 196-201 (2006)]. Similar data were obtained with human sputum samples and bronchial alveolar washing fluid (BALF). Here, HME on activated macrophages was able to be detected and quantified: HME amount COPD patient/smoker >COPD patient/former smoker >former smoker >nonsmoker [Patterns of airway inflammation and MMP-12 expression in smokers and ex-smokers with COPD, Babusyte et al., Respir. Res. 8, 81-90 (2007)].

An inflammatory lung disease similar to COPD in a certain way is interstitial lung disease (ILD), in particular here the manifestation as idiopathic pulmonary fibrosis (IPF) and sarcoidosis [Commonalities between the pro fibrotic mechanisms in COPD and IPF, L. A. Murray, Pulm. Pharmacol. Therap. 25, 276-280 (2012); The pathogenesis of COPD and IPF: distinct horns of the same devil?, Chilosi et al., Respir. Res. 13:3 (2012)]. Here too, the homeostasis of the extracellular matrix is disturbed. Data from genome-wide association studies suggest a particular role of HME in disease incidence of such fibrotic diseases [Gene Expression Profiling Identifies MMP-12 and ADAMDEC 1 as Potential Pathogenic Mediators of Pulmonary Sarcoidosis, Crouser et al., Am. J. Respir. Crit. Care Med. 179, 929-938 (2009); Association of a Functional Polymorphism in the Matrix Metalloproteinase-12 Promoter Region with Systemic Sclerosis in an Italian Population, Manetti et al., J. Rheumatol. 37, 1852-1857 (2010); Increased serum levels and tissue expression of matrix metalloproteinase-12 in patients with systemic sclerosis: correlation with severity of skin and pulmonary fibrosis and vascular damage, Manetti et al., Ann. Rheum. Dis. 71, 1064-1070 (2012)].

Moreover, there is further preclinical evidence of a decisive role of HME in ischaemic-inflammatory disease processes [Macrophage Metalloelastase (MMP-12) Deficiency Mitigates Retinal Inflammation and Pathological Angiogenesis in Ischemic Retinopathy, Li et al., PLoS ONE 7 (12), e52699 (2012)]. A significantly higher MMP-12 expression is also known in ischaemic kidney injuries, as is the participation of MMP-12 in further inflammatory kidney diseases [JNK signalling in human and experimental renal ischaemia/reperfusion injury, Kanellis et al., Nephrol. Dial. Transplant. 25, 2898-2908 (2010); Macrophage Metalloelastase as a Major Factor for Glomerular Injury in Anti-Glomerular Basement Membrane Nephritis, Kaneko et al., J. Immun 170, 3377-3385 (2003); Role for Macrophage Metalloelastase in Glomerular Basement Membrane Damage Associated with Alport Syndrome, Rao et al., Am. J. Pathol. 169, 32-46 (2006); Differential regulation of metzincins in experimental chronic renal allograft rejection: Potential markers and novel therapeutic targets, Berthier et al., Kidney Int. 69, 358-368 (2006); Macrophage infiltration and renal damage are independent of Matrix Metalloproteinase 12 (MMP-12) in the obstructed kidney, Abraham et al., Nephrology 17, 322-329 (2012)].

The object of the present invention was therefore the identification and provision of new substances which act as potent, selective and specific inhibitors of human macrophage elastase (HME/MMP-12) and as such are suitable for the treatment and/or prevention in particular of diseases of the respiratory tract, the lung and the cardiovascular system.

The patent applications WO 96/15096-A1, WO 97/43237-A1, WO 97/43238-A1, WO 97/43239-A1, WO 97/43240-A1, WO 97/43245-A1 and WO 97/43247-A1 disclose 4-aryl- and 4-biaryl-substituted 4-oxobutanoic acid derivatives with an inhibitory activity towards MMP-2, MMP-3, MMP-9 and, to a lesser extent, MMP-1; on account of this activity profile, these compounds were considered to be suitable particularly for the treatment of osteoarthritis, rheumatoid arthritis and tumour diseases. WO 98/09940-A1 and WO 99/18079-A1 disclose further biarylbutanoic acid derivatives as inhibitors of MMP-2, MMP-3 and/or MMP-13 which are suitable for treating a wide variety of diseases. WO 00/40539-A1 claims the use of 4-biaryl-4-oxobutanoic acids for treating lung and respiratory tract diseases, based on a differently marked inhibition of MMP-2, MMP-3, MMP-8, MMP 9, MMP-12 and MMP-13 by these compounds. Furthermore, WO 2012/014114-A1 describes 3-hydroxypropionic acid derivatives and WO 2012/038942-A1 describes oxy- or sulphonylacetic acid derivatives as dual MMP 9/12 inhibitors.

Against the background of the object described above, however, it has been found that these MMP inhibitors from the prior art often have disadvantages, such as in particular an inadequate inhibitory potency towards MMP-12, an inadequate selectivity for MMP-12 compared to other MMPs and/or a limited metabolic stability.

Further arylalkanecarboxylic acid derivatives are described in WO 2004/092146-A2, WO 2004/099168-A2, WO 2004/099170-A2, WO 2004/099171-A2, WO 2006/050097-A1 and WO 2006/055625-A2 as inhibitors of protein-tyrosine-phosphatase 1B (PTP-1B) for the treatment of diabetes, cancer diseases and neurodegenerative diseases.

Surprisingly, it has now been found that certain 2,5-disubstituted cyclopentanecarboxylic acid derivatives have a significantly improved profile as regards their activity strength and selectivity towards human macrophage elastase (HME/hMMP-12) compared to the compounds known from the prior art. Moreover, the compounds according to the invention exhibit a low nonspecific binding to blood plasma constituents such as albumin and, moreover, they have a low in vivo clearance and a good metabolic stability. This profile of properties overall suggests, for the compounds according to the invention, a low dosability and—as a result of the more targeted mode of action—a reduced risk of the appearance of undesired side effects during therapy.

The compounds according to the invention are moreover characterized by a significant inhibitory activity and selectivity towards the orthologous MMP-12 peptidases of rodents, such as MMP-12 of the mouse (also referred to as murine macrophage elastase, MME) and MMP-12 of the rat. This facilitates a more comprehensive preclinical evaluation of the substances in a variety of established animal models of the diseases described above.

The present invention provides the compounds (1S,2S,5R)-2-[4-(benzyloxy)benzoyl]-5-[(4-oxo-1,2,3-benzotriazin-3(4H)-yl)methyl]cyclopentanecarboxylic acid of the formula (I-A) and (1R,2R,5S)-2-[4-(benzyloxy)benzoyl]-5-[(4-oxo-1,2,3-benzotriazin-3(4H)-yl)methyl]cyclopentanecarboxylic acid of the formula (I-B)

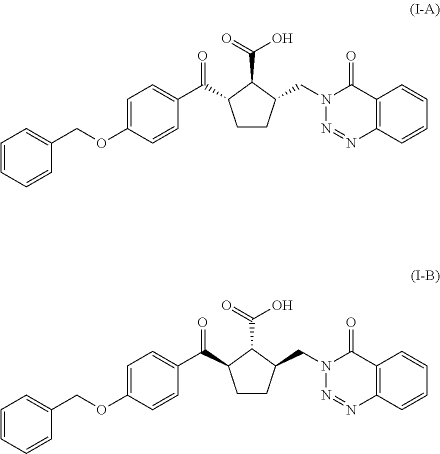 [SMILES: O=C(O)[C@H]1[C@H](Cn2nnc3ccccc3c2=O)CC[C@@H]1C(=O)c1ccc(OCc2ccccc2)cc1 ; O=C(O)[C@@H]1[C@@H](Cn2nnc3ccccc3c2=O)CC[C@H]1C(=O)c1ccc(OCc2ccccc2)cc1]

in isolated, enantiomerically pure form or in the form of a mixture of these compounds, and also the salts, solvates and solvates of the salts of these compounds or of their mixture.

A particular embodiment of the present invention relates to the compounds of the formula (I-A) and (I-B) in the form of their racemic mixture or as salt, solvate or solvate of a salt of this racemic mixture.

In the context of the present invention, preference is given to the compound (1S,2S,5R)-2-[4-(benzyloxy)benzoyl]-5-[(4-oxo-1,2,3-benzotriazin-3(4H)-yl)methyl]cyclopentanecarboxylic acid of the formula (I-A)

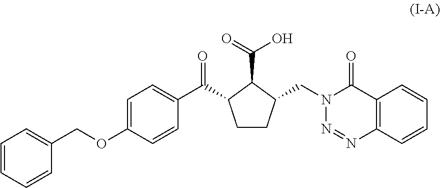 [SMILES: O=C(O)[C@H]1[C@H](Cn2nnc3ccccc3c2=O)CC[C@@H]1C(=O)c1ccc(OCc2ccccc2)cc1]

in enantiomerically pure form or a salt, solvate or solvate of a salt thereof.

In the context of the present invention, the term “enantiomerically pure” is to be understood as meaning that the compound in question with respect to the absolute configuration of the chiral centres is present in an enantiomeric excess of more than 95%, preferably more than 98%. The enantiomeric excess, ee, is calculated here by evaluating an HPLC analysis chromatogram on a chiral phase using the formula below:

$$ee = \vert \frac{Enantiomer 1 ( area per cent ) - Enantiomer 2 ( area per cent )}{Enantiomer 1 ( area per cent ) + Enantiomer 2 ( area per cent )} \vert \times 100 \% .$$

Hereinbelow, the compounds of the formula (I-A) and (I-B) in the narrower sense, and the mixtures of these compounds and the salts, solvates and solvates of the salts of these compounds and their mixtures in the further sense are referred to in summary as “compounds according to the invention”.

In the context of the present invention, the salts are preferably physiologically acceptable salts. Also encompassed are salts which are not themselves suitable for pharmaceutical applications but can be used, for example, for the isolation, purification or storage of the compounds according to the invention.

Physiologically acceptable salts of the compounds according to the invention include in particular the salts derived from conventional bases, by way of example and with preference alkali metal salts (e.g. sodium and potassium salts), alkaline earth metal salts (e.g. calcium and magnesium salts), zinc salts and ammonium salts derived from ammonia or organic amines having 1 to 16 carbon atoms, by way of example and with preference ethylamine, diethylamine, triethylamine, N,N-ethyldiisopropylamine, monoethanolamine, diethanolamine, triethanolamine, tromethamine, dimethylaminoethanol, diethylaminoethanol, choline, procaine, dicyclohexylamine, dibenzylamine, N-methylmorpholine, N-methylpiperidine, arginine, lysine and 1,2-ethylenediamine.

Solvates in the context of the invention are described as those forms of the compounds according to the invention which form a complex in the solid or liquid state by coordination with solvent molecules. Hydrates are a specific form of the solvates in which the coordination is with water. Solvates preferred in the context of the present invention are hydrates.

The present invention also encompasses all suitable isotopic variants of the compounds according to the invention. An isotopic variant of a compound according to the invention is understood here as meaning a compound in which at least one atom within the compound according to the invention has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen and oxygen, such as ²H (deuterium), ³H (tritium), ¹³C, ¹⁵N, ¹⁷O and ¹⁸O. Particular isotopic variants of a compound according to the invention, especially those in which one or more radioactive isotopes have been incorporated, may be beneficial, for example, for the examination of the mechanism of action or of the active compound distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example to an extension of the half-life in the body or to a reduction in the active dose required; such modifications of the compounds according to the invention may therefore in some cases also constitute a preferred embodiment of the present invention. Isotopic variants of the compounds according to the invention can be prepared by generally customary processes known to those skilled in the art, for example by the methods described below and the procedures reported in the working examples, by using corresponding isotopic modifications of the particular reagents and/or starting compounds therein.

In addition, the present invention also encompasses prodrugs of the compounds according to the invention. The term “prodrugs” refers here to compounds which may themselves be biologically active or inactive, but are converted while present in the body, for example by a metabolic or hydrolytic route, to compounds according to the invention.

In particular, the present invention encompasses, as prodrugs, hydrolysable ester derivatives of the carboxylic acids of the formula (I-A) and (I-B) according to the invention. These are to be understood as meaning esters which can be hydrolysed to the free carboxylic acids, as the main biologically active compounds, in physiological media, under the conditions of the biological tests described hereinbelow and in particular in vivo by enzymatic or chemical routes. (C₁-C₄)-Alkyl esters, in which the alkyl group can be straight-chain or branched, are preferred as such esters. Particular preference is given to methyl, ethyl or tert-butyl esters.

The invention furthermore provides a process for preparing the compounds according to the invention of the formulae (I-A) and (I-B), characterized in that exo-2-(trimethylsilyl)ethyl 2-oxobicyclo[2.2.1]heptane-7-carboxylate of the formula (II)

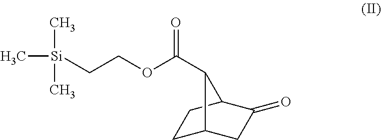 [SMILES: C[Si](C)(C)CCOC(=O)C1C2CCC1C(=O)C2]

is reacted with a phenyl-Grignard compound of the formula (III)

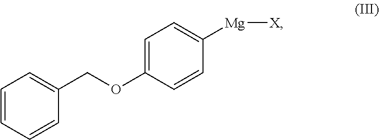

in which X is chlorine, bromine or iodine,

to give the adduct of the formula (IV)

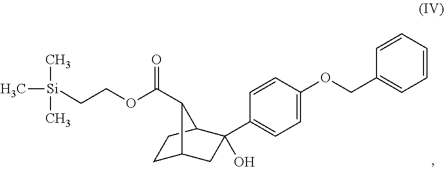 [SMILES: C[Si](C)(C)CCOC(=O)C1C2CCC1C(O)(c1ccc(OCc3ccccc3)cc1)C2]

then the hydroxy group is eliminated via the mesylate produced in situ of the formula (V)

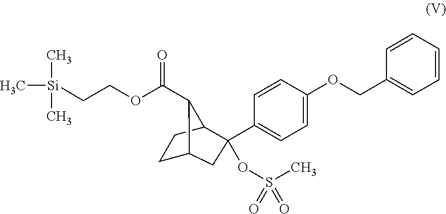 [SMILES: C[Si](C)(C)CCOC(=O)C1C2CCC1C(OS(C)(=O)=O)(c1ccc(OCc3ccccc3)cc1)C2]

to give the olefin of the formula (VI)

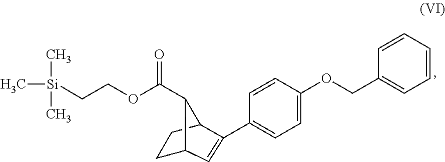 [SMILES: C[Si](C)(C)CCOC(=O)C1C2C=C(c3ccc(OCc4ccccc4)cc3)C1CC2]

then oxidation is carried out with N-methylmorpholine-N-oxide together with osmium tetroxide as catalyst to give the cis-1,2-diol of the formula (VII)

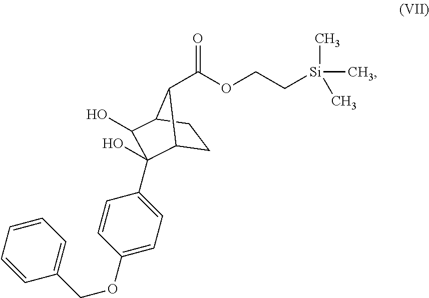 [SMILES: C[Si](C)(C)CCOC(=O)C1C2CCC1C(O)(c1ccc(OCc3ccccc3)cc1)C2O]

then this bicyclic diol is cleaved with the help of lead tetraacetate or sodium periodate to give the racemic mixture of the 2-benzoyl-5-formylcyclopentanecarboxylic acid esters (VIII-A) and (VIII-B)

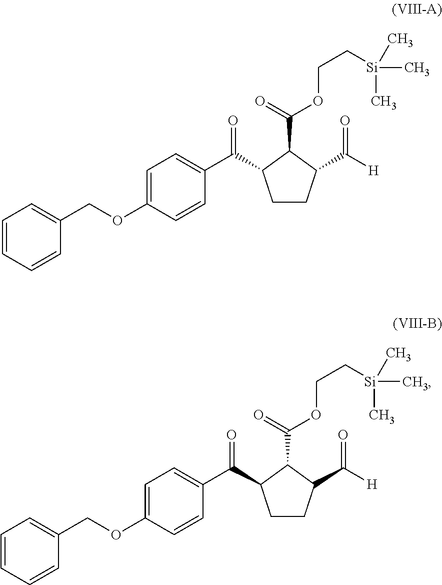

this mixture is reduced with sodium borohydride to give the racemic mixture of the hydroxymethyl compounds (IX-A) and (IX-B)

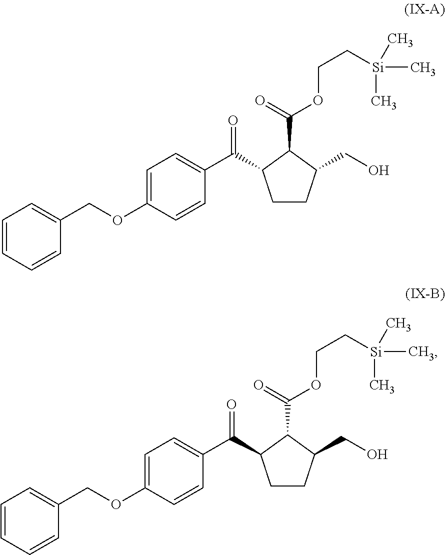 [SMILES: C[Si](C)(C)CCOC(=O)[C@H]1[C@H](CO)CC[C@@H]1C(=O)c1ccc(OCc2ccccc2)cc1 ; C[Si](C)(C)CCOC(=O)[C@@H]1[C@@H](CO)CC[C@H]1C(=O)c1ccc(OCc2ccccc2)cc1]

then reaction is carried out with 1,2,3-benzotriazin-4(3H)-one of the formula (X)

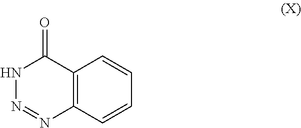 [SMILES: O=c1[nH]nnc2ccccc12]

in the presence of an alkyl- or arylphosphane and an azodicarboxylate to give the racemic mixture of the benzotriazinone derivatives (XI-A) and (XI-B)

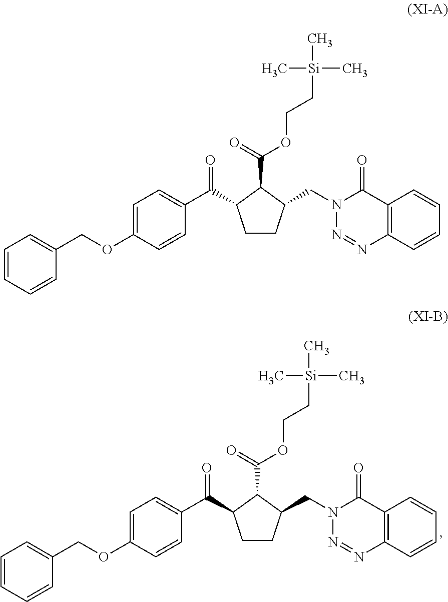 [SMILES: C[Si](C)(C)CCOC(=O)[C@H]1[C@H](Cn2nnc3ccccc3c2=O)CC[C@@H]1C(=O)c1ccc(OCc2ccccc2)cc1 ; C[Si](C)(C)CCOC(=O)[C@@H]1[C@@H](Cn2nnc3ccccc3c2=O)CC[C@H]1C(=O)c1ccc(OCc2ccccc2)cc1]

and finally the 2-(trimethylsilyl)ethyl ester group is cleaved off with the help of an acid or of a fluoride reagent to give the racemic mixture of the cyclopentanecarboxylic acids according to the invention (I-A) and (I-B)

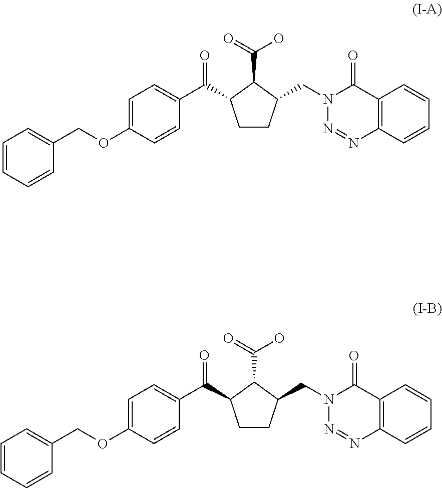 [SMILES: O=C(O)[C@H]1[C@H](Cn2nnc3ccccc3c2=O)CC[C@@H]1C(=O)c1ccc(OCc2ccccc2)cc1 ; O=C(O)[C@@H]1[C@@H](Cn2nnc3ccccc3c2=O)CC[C@H]1C(=O)c1ccc(OCc2ccccc2)cc1]

and optionally the resulting mixture of the compounds (I-A) and (I-B) is separated into the enantiomerically pure compounds and/or converted with the corresponding (i) solvents and/or (ii) bases to the solvates, salts and/or solvates of the salts.

The Grignard reaction (II)+(III)→(IV) is carried out under customary conditions in an ethereal solvent such as diethyl ether or tetrahydrofuran in a temperature range from −20° C. to +25° C.

By reacting the tertiary alcohol (IV) with methanesulphonyl chloride in the presence of an excess of a customary amine base, such as, for example, triethylamine, N,N-diisopropylethylamine or pyridine, the mesylate (V) is produced, which eliminates under the reaction conditions in situ to the olefin (VI). The reaction (IV)→(V)→(VI) takes place under customary conditions in a chlorohydrocarbon, such as dichloromethane or chloroform, as an inert solvent in a temperature range from −10° C. to +25° C. The transformation (IV)→(VI) (dehydration) can alternatively also be effected by treatment of (IV) with phosphorus oxychloride or thionyl chloride in the presence of excess pyridine [cf. e.g. C. A. Grob et al., Helv. Chim. Acta 66 (8), 2656-2665 (1983)].

The bis-hydroxylation of the olefin (VI) to the cis-1,2-diol (VII) is effected according to known methodology by reaction with N-methylmorpholine N-oxide (NMO) in the presence of catalytic osmium tetroxide (as commercially available solution in tert-butanol or water). The reaction is usually carried out in a mixture of tetrahydrofuran and/or acetone with water in a temperature range from 0° C. to +25° C.

Suitable oxidizing agents for the subsequent diol cleavage (VII)→(VIII-A)/(VIII-B) are in particular lead tetraacetate or sodium periodate. The reaction with lead tetraacetate is preferably carried out in an alcoholic solvent such as methanol and in a temperature range from −20° C. to +25° C. The reaction with sodium periodate generally takes place in a mixture of tetrahydrofuran and/or acetone with water in a temperature range from 0° C. to +25° C. When using sodium periodate for the diol cleavage, the transformation (VI)→(VII)→(VIII-A)/(VIII-B) can also be carried out in a “one-pot process”, i.e. without interim isolation of (VII).

The reduction of the formyl compound (VIII-A)/(VIII-B) to the primary alcohol (IX-A)/(IX-B) takes place by a known method by reaction with sodium borohydride in an alcoholic solvent such as methanol or ethanol in a temperature range from 0° C. to +25° C.

The reaction (IX-A)/(IX-B)+(X)→(XI-A)/(XI-B) is carried out under the customary conditions of a “Mitsunobu reaction” in the presence of a phosphine and an azodicarboxylate [see e.g. D. L. Hughes, Org. Reactions 42, 335 (1992); D. L. Hughes, Org. Prep. Proced. Int. 28 (2), 127 (1996)]. Of suitability as phosphine component are, for example, triphenylphosphine, tri-n-butylphosphine, 1,2-bis(diphenylphosphino)ethane (DPPE), diphenyl(2-pyridyl)phosphine, (4-dimethylaminophenyl)diphenylphosphine or tris(4-dimethylaminophenyl)phosphine, and an azodicarboxylate that can be used is, for example, diethyl azodicarboxylate (DEAD), diisopropyl azodicarboxylate (DIAD), di-tert-butyl azodicarboxylate, N,N,N′N′-tetramethylazodicarboxamide (TMAD), 1,1′-(azodicarbonyl)dipiperidine (ADDP) or 4,7-dimethyl-3,5,7-hexahydro-1,2,4,7-tetrazocine-3,8-dione (DHTD). Preferably, tri-n-butylphosphine in conjunction with diethyl azodicarboxylate (DEAD) is used here. The inert solvent used is preferably tetrahydrofuran, toluene or a mixture of the two. The reaction is generally carried out in a temperature range of from −20° C. to +40° C., preferably from 0° C. to +25° C.

The cleaving off of the 2-(trimethylsilyl)ethyl ester group in the process step (XI-A)/(XI-B)→(I-A)/(I-B) takes place in accordance with customary methods either with the help of a strong acid, such as in particular trifluoroacetic acid, in an inert solvent such as dichloromethane or with the help of a fluoride, such as in particular tetra-n-butylammonium fluoride (TBAF), in an ethereal solvent such as tetrahydrofuran. The ester cleavage is generally carried out in a temperature range of from −20° C. to +25° C.

The mixtures of the compounds according to the invention can optionally, according to suitability, also be separated into the enantiomerically pure compounds already at the stage of the intermediates (IX-A)/(IX-B) or (XI-A)/(XI-B), which are then further reacted in separate form according to the reaction sequence described above. Such a separation of stereoisomers can be carried out by customary methods known to the person skilled in the art. In the context of the present invention, preferably chromatographic methods on chiral separation phases are used; in the case of the carboxylic acids (I-A)/(I-B), a separation can alternatively also take place via diastereomeric salts with the help of chiral bases.

The preparation of exo-2-(trimethylsilyl)ethyl 2-oxobicyclo[2.2.1]heptane-7-carboxylate (II) is described [see WO 96/15096, Example 360/Stage 1 and further literature cited therein]. The compounds of the formulae (III) and (X) are either commercially available or described as such in the literature, or they can be prepared in a way obvious to the person skilled in the art, in analogy to methods published in the literature. Numerous detailed procedures can also be found in the Experimental Part, in the section on the preparation of the starting compounds and intermediates.

The preparation of the compounds according to the invention is summarized in the reaction scheme below:

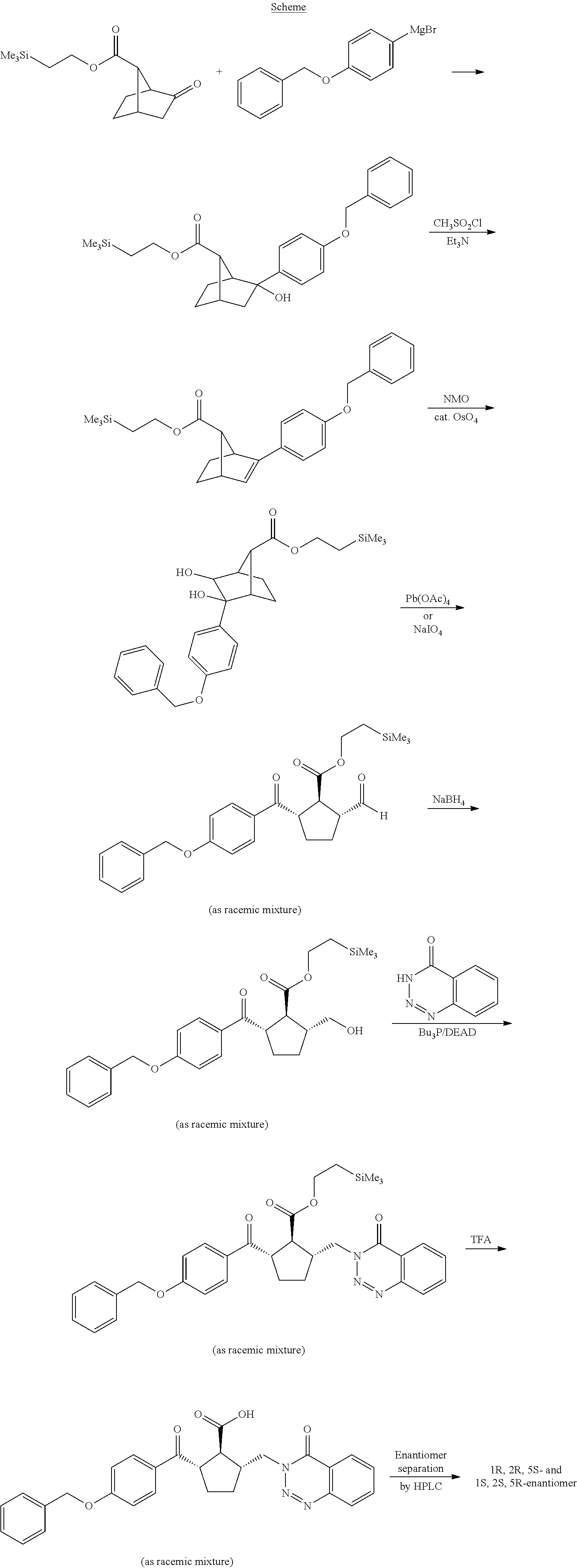

The compounds according to the invention have valuable pharmacological properties and can be used for prevention and treatment of diseases in humans and animals.

The compounds according to the invention are potent, nonreactive and selective inhibitors of human macrophage elastase (HME/hMMP-12), which, compared to the compounds known from the prior art, have a significantly improved profile as regards the combination of activity strength and selectivity. Moreover, the compounds according to the invention exhibit a high HME-inhibitory activity even under the test conditions of a potentially competing nonspecific binding to blood plasma constituents such as albumin. Moreover, the compounds according to the invention have a low in vivo clearance and a good metabolic stability. This profile of properties overall suggests, for the compounds according to the invention, a low dosability and—as a result of the more targeted mode of action—a reduced risk of the appearance of undesired side effects during therapy.

The compounds according to the invention are therefore suitable to a particular extent for the treatment and/or prevention of diseases and pathological processes, in particular those in which macrophage elastase (HME/hMMP-12) is involved in the course of an infectious or noninfectious inflammatory event and/or tissue or vascular remodelling.

In the context of the present invention, these include in particular diseases of the respiratory tract and the lungs, such as chronic obstructive pulmonary disorder (COPD), asthma and the group of interstitial lung diseases (ILD), and also diseases of the cardiovascular system, such as arteriosclerosis and aneurysms.

Manifestations of chronic obstructive pulmonary disease (COPD) include in particular pulmonary emphysema, e.g. pulmonary emphysema induced by cigeratte smoke, chronic bronchitis (CB), pulmonary hypertension in COPD (PH-COPD), bronchiectasis (BE) and combinations thereof, particularly in acutely exacerbating stages of the disease (AE-COPD).

Manifestations of asthma include asthmatic diseases of differing degrees of severity with intermittent or persistent course, such as refractory asthma, bronchial asthma, allergic asthma, intrinsic asthma, extrinsic asthma and asthma induced by medicaments or dust.

The group of interstitial pulmonary diseases (ILD) include idiopathic pulmonary fibrosis (IPF), pulmonary sarcoidosis and acute interstitial pneumonia, nonspecific interstitial pneumonias, lymphoid interstitial pneumonias, respiratory bronchiolitis with interstitial pulmonary disease, cryptogenic organizing pneumonias, desquamative interstitial pneumonias and non-classifiable idiopathic interstitial pneumonias, also granulomatous interstitial pulmonary diseases, interstitial pulmonary diseases of known origin and other interstitial pulmonary diseases of unknown origin.

The compounds according to the invention can also be used for the treatment and/or prevention of further diseases of the respiratory tracts and the lungs, such as e.g. pulmonary arterial hypertension (PAH) and other forms of pulmonary hypertension (PH), bronchiolitis obliterans-syndrome (BOS), acute respiratory tract syndrome (ARDS), acute lung injury (ALI), alpha-1-antitrypsin deficiency (AATD) and cystic fibrosis (CF), of various forms of bronchitis (chronic bronchitis, infectious bronchitis, eosinophilic bronchitis), of bronchiectasis, pneumonia, farmer's lung and related diseases, infectious and noninfectious cough and cold illnesses (chronic inflammatory coughs, iatrogenic coughs), nasal mucosa inflammations (including medicamentous rhinitis, vasomotor rhinitis and seasonal allergic rhinitis, e.g. hayfever) and of polyps.

The group of diseases of the cardiovascular system include in the context of the present invention in particular arteriosclerosis and its secondary diseases, such as e.g. stroke in the case of arteriosclerosis of the neck arteries (carotid arteriosclerosis), cardiac infarction in the case of arteriosclerosis of the coronary artery, peripheral arterial occlusive disease (pAOD) as a consequence of arteriosclerosis of arteries of the legs, and also aneurysms, in particular aneurysms of the aorta, e.g. as a consequence of arteriosclerosis, high blood pressure, injuries and inflammations, infections (e.g. in the case of rheumatic fever, syphilis, lyme borreliosis), inherited connective tissue weaknesses (e.g. in the case of Marfan syndrome and Ehlers-Danlos syndrome) or as a consequence of a volume load on the aorta in the case of inherited heart defects with right-left shunt or a shunt-dependent perfusion of the lungs, and also aneurysms at coronary arteries in the course of a disease from Kawasaki syndrome and in areas of the brain in patients with an inherited defect of the aortic valve.

In addition, the compounds according to the invention can be used for the treatment and/or prevention of further cardiovascular disorders such as, for example, high blood pressure (hypertension), heart failure, coronary heart disease, stable and unstable angina pectoris, renal hypertension, peripheral and cardiac vascular disorders, arrhythmias, atrial and ventricular arrhythmias and impaired conduction such as, for example, atrioventricular blocks of degrees I-III, supraventricular tachyarrhythmia, atrial fibrillation, atrial flutter, ventricular fibrillation, ventricular flutter, ventricular tachyarrhythmia, Torsade de pointes tachycardia, atrial and ventricular extrasystoles, AV junctional extrasystoles, sick sinus syndrome, syncopes, AV-nodal re-entry tachycardia, Wolff-Parkinson-White syndrome, acute coronary syndrome (ACS), autoimmune cardiac disorders (pericarditis, endocarditis, valvolitis, aortitis, cardiomyopathies), boxer cardiomyopathy, shock such as cardiogenic shock, septic shock and anaphylactic shock, furthermore for the treatment and/or prevention of thromboembolic disorders and ischaemias such as myocardial ischaemia, cardiac hypertrophy, transient and ischaemic attacks, preeclampsia, inflammatory cardiovascular disorders, spasms of the coronary arteries and peripheral arteries, oedema formation such as, for example, pulmonary oedema, cerebral oedema, renal oedema or oedema caused by heart failure, peripheral circulatory disturbances, reperfusion damage, arterial and venous thromboses, microalbuminuria, myocardial insufficiency, endothelial dysfunction, micro- and macrovascular damage (vasculitis), and also to prevent restenoses, for example after thrombolysis therapies, percutaneous transluminal angioplasties (PTA), percutaneous transluminal coronary angioplasties (PTCA), heart transplants and bypass operations.

In the context of the present invention, the term “heart failure” encompasses both acute and chronic forms of heart failure, and also specific or related disease types thereof, such as acute decompensated heart failure, right heart failure, left heart failure, global failure, ischaemic cardiomyopathy, dilated cardiomyopathy, hypertrophic cardiomyopathy, idiopathic cardiomyopathy, congenital heart defects, heart valve defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders and diastolic and systolic heart failure.

The compounds according to the invention are also suitable for the treatment and/or prevention of renal disorders, in particular renal insufficiency and kidney failure. In the context of the present invention, the terms “renal insufficiency” and “kidney failure” encompass both acute and chronic manifestations thereof and also underlying or related renal disorders such as renal hypoperfusion, intradialytic hypotension, obstructive uropathy, glomerulopathies, glomerulonephritis, acute glomerulonephritis, glomerulosclerosis, tubulointerstitial diseases, nephropathic disorders such as primary and congenital kidney disease, nephritis, immunological kidney disorders such as kidney transplant rejection and Alport's syndrome, immunocomplex-induced kidney disorders, nephropathy induced by toxic substances, nephropathy induced by contrast agents, diabetic and non-diabetic nephropathy, pyelonephritis, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerulae and arterioles, tubular dilatation, hyperphosphataemia and/or need for dialysis. The present invention also comprises the use of the compounds according to the invention for the treatment and/or prevention of sequelae of renal insufficiency, for example hypertension, pulmonary oedema, heart failure, uraemia, anaemia, electrolyte disturbances (for example hyperkalaemia, hyponatraemia) and disturbances in bone and carbohydrate metabolism.

In addition, the compounds according to the invention are suitable for the treatment and/or prevention of disorders of the urogenital system such as, for example, benign prostate syndrome (BPS), benign prostate hyperplasia (BPH), benign prostate enlargement (BPE), bladder outlet obstruction (BOO), lower urinary tract syndromes (LUTS), neurogenic overactive bladder (OAB), incontinence such as, for example, mixed urinary incontinence, urge urinary incontinence, stress urinary incontinence or overflow urinary incontinence (MUI, UUI, SUI, OUI), pelvic pain, and also erectile dysfunction and female sexual dysfunction.

In addition, the compounds according to the invention have antiinflammatory action and can therefore be used as antiinflammatory agents for treatment and/or prevention of sepsis (SIRS), multiple organ failure (MODS, MOF), inflammatory disorders of the kidney, chronic intestinal inflammations (IBD, Crohn's disease, ulcerative colitis), pancreatitis, peritonitis, cystitis, urethritis, prostatitis, epidimytitis, oophoritis, salpingitis, vulvovaginitis, rheumatoid disorders, inflammatory disorders of the central nervous system, multiple sclerosis, infammatory skin disorders and inflammatory eye disorders.

Furthermore, the compounds according to the invention are suitable for treatment and/or prevention of fibrotic disorders of the internal organs, for example the lung, the heart, the kidney, the bone marrow and in particular the liver, and also dermatological fibroses and fibrotic eye disorders. In the context of the present invention, the term “fibrotic disorders” includes in particular disorders such as hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis, peritoneal fibrosis and similar fibrotic disorders, scleroderma, morphoea, keloids, hypertrophic scarring, naevi, diabetic retinopathy, proliferative vitroretinopathy and disorders of the connective tissue (for example sarcoidosis). The compounds according to the invention can likewise be used for promoting wound healing, for controlling postoperative scarring, for example as a result of glaucoma operations and cosmetically for ageing or keratinized skin.

The compounds according to the invention can also be employed for the treatment and/or prevention of anaemias such as haemolytic anaemias, in particular haemoglobinopathies such as sickle cell anaemia and thalassaemias, megaloblastic anaemias, iron deficiency anaemias, anaemias owing to acute blood loss, displacement anaemias and aplastic anaemias.

Moreover, the compounds according to the invention are suitable for the treatment of cancers such as, for example, skin cancer, brain tumours, breast cancer, bone marrow tumours, leukaemias, liposarcomas, carcinomas of the gastrointestinal tract, of the liver, the pancreas, the lung, the kidney, the ureter, the prostate and the genital tract and also of malignant tumours of the lymphoproliferative system, for example Hodgkin and Non-Hodgkin lymphoma.

In addition, the compounds according to the invention can be used for the treatment and/or prevention of impaired lipid metabolism and dyslipidaemias (hypolipoproteinaemia, hypertriglyceridaemias, hyperlipidaemia, combined hyperlipidaemias, hypercholesterolaemia, abetalipoproteinaemia, sitosterolaemia), xanthomatosis, Tangier disease, adiposity, obesity, metabolic disorders (metabolic syndrome, hyperglycaemia, insulin-dependent diabetes, non-insulin-dependent diabetes, gestational diabetes, hyperinsulinaemia, insulin resistence, glucose intolerance and diabetic sequelae, such as retinopathy, nephropathy and neuropathy), of disorders of the gastrointestinal tract and the abdomen (glossitis, gingivitis, periodontitis, oesophagitis, eosinophilic gastroenteritis, mastocytosis, Crohn's disease, colitis, proctitis, anus pruritis, diarrhoea, coeliac disease, hepatitis, hepatic fibrosis, cirrhosis of the liver, pancreatitis and cholecystitis), of disorders of the central nervous system and neurodegenerative disorders (stroke, Alzheimer's disease, Parkinson's disease, dementia, epilepsy, depressions, multiple sclerosis), immune disorders, thyroid disorders (hyperthyreosis), skin disorders (psoriasis, acne, eczema, neurodermitis, various forms of dermatitis, such as, for example, dermatitis abacribus, actinic dermatitis, allergic dermatitis, ammonia dermatitis, facticial dermatitis, autogenic dermatitis, atopic dermatitis, dermatitis calorica, dermatitis combustionis, dermatitis congelationis, dermatitis cosmetica, dermatitis escharotica, exfoliative dermatitis, dermatitis gangraenose, stasis dermatitis, dermatitis herpetiformis, lichenoid dermatitis, dermatitis linearis, dermatitis maligna, medicinal eruption dermatitis, dermatitis palmaris and plantaris, parasitic dermatitis, photoallergic contact dermatitis, phototoxic dermatitis, dermatitis pustularis, seborrhoeic dermatitis, sunburn, toxic dermatitis, Meleney's ulcer, dermatitis veneata, infectious dermatitis, pyogenic dermatitis and rosacea-like dermatitis, and also keratitis, bullosis, vasculitis, cellulitis, panniculitis, lupus erythematosus, erythema, lymphomas, skin cancer, Sweet syndrome, Weber-Christian syndrome, scar formation, wart formation, chilblains), of inflammatory eye diseases (saccoidosis, blepharitis, conjunctivitis, iritis, uveitis, chorioiditis, ophthalmitis), viral diseases (caused by influenza, adeno and corona viruses, such as, for example, HPV, HCMV, HIV, SARS), of disorders of the skeletal bone and the joints and also the skeletal muscle (multifarious forms of arthritis, such as, for example, arthritis alcaptonurica, arthritis ankylosans, arthritis dysenterica, arthritis exsudativa, arthritis fungosa, arthritis gonorrhoica, arthritis mutilans, arthritis psoriatica, arthritis purulenta, arthritis rheumatica, arthritis serosa, arthritis syphilitica, arthritis tuberculosa, arthritis urica, arthritis villonodularis pigmentosa, atypical arthritis, haemophilic arthritis, juvenile chronic arthritis, rheumatoid arthritis and metastatic arthritis, furthermore Still syndrome, Felty syndrome, Sjörgen syndrome, Clutton syndrome, Poncet syndrome, Pott syndrome and Reiter syndrome, multifarious forms of arthropathies, such as, for example, arthropathia deformans, arthropathia neuropathica, arthropathia ovaripriva, arthropathia psoriatica and arthropathia tabica, systemic scleroses, multifarious forms of inflammatory myopathies, such as, for example, myopathie epidemica, myopathie fibrosa, myopathie myoglobinurica, myopathie ossificans, myopathie ossificans neurotica, myopathie ossificans progressiva multiplex, myopathie purulenta, myopathie rheumatica, myopathie trichinosa, myopathie tropica and myopathie typhosa, and also the Gunther syndrome and the Münchmeyer syndrome), of inflammatory changes of the arteries (multifarious forms of arteritis, such as, for example, endarteritis, mesarteritis, periarteritis, panarteritis, arteritis rheumatica, arteritis deformans, arteritis temporalis, arteritis cranialis, arteritis gigantocellularis and arteritis granulomatosa, and also Horton syndrome, Churg-Strauss syndrome and Takayasu arteritis), of Muckle-Well syndrome, of Kikuchi disease, of polychondritis, dermatosclerosis and also other disorders having an inflammatory or immunological component, such as, for example, cataract, cachexia, osteoporosis, gout, incontinence, lepra, Sezary syndrome and paraneoplastic syndrome, for rejection reactions after organ transplants and for wound healing and angiogenesis in particular in the case of chronic wounds.

On account of their property profile, the compounds according to the invention are suitable in particular for the treatment and/or prevention of diseases of the respiratory tract and of the lung, primarily chronic obstructive pulmonary disorder (COPD), here in particular lung emphysema, chronic bronchitis (CB), pulmonary hypertension in COPD (PH-COPD) and bronchiectasis (BE), and also of combinations of these types of illnesses, particularly in acutely exacerbating stages of COPD disease (AE COPD), furthermore of asthma and of interstitial lung diseases, here in particular idiopathic pulmonary fibrosis (IPF) and pulmonary sarcoidosis, of diseases of the cardiovascular system, in particular of arteriosclerosis, specifically of carotid arteriosclerosis, and also viral myocarditis, cardiomyopathy and aneurysms, including their sequelae such as stroke, myocardial infarction and peripheral arterial occlusive disease (pAVK), and also of chronic kidney diseases and Alport's syndrome.

The above-mentioned, well-characterized diseases in humans can also occur with a comparable aetiology in other mammals and can likewise be treated there with the compounds of the present invention.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

The present invention further provides for the use of the compounds according to the invention for treatment and/or prevention of disorders, especially the aforementioned disorders.

The present invention further provides for the use of the compounds according to the invention for producing a medicament for the treatment and/or prevention of disorders, in particular the disorders mentioned above.

The present invention further provides a medicament comprising at least one of the compounds according to the invention, for the treatment and/or prevention of disorders, in particular the disorders mentioned above.

The present invention further provides for the use of the compounds according to the invention in a method for treatment and/or prevention of disorders, in particular the disorders mentioned above.

The present invention further provides a method for treatment and/or prevention of disorders, in particular the disorders mentioned above, using an effective amount of at least one of the compounds according to the invention.

The compounds according to the invention can be used alone or, if required, in combination with one or more other pharmacologically active substances, provided that this combination does not lead to undesirable and unacceptable side effects. The present invention furthermore therefore provides medicaments containing at least one of the compounds according to the invention and one or more further active compounds, in particular for treatment and/or prevention of the abovementioned disorders. Preferred examples of active compounds suitable for combinations include:
anti-obstructive/bronchodilatory agents as used, for example, for the therapy of chronic obstructive pulmonary disease (COPD) or bronchial asthma, by way of example and with preference from the group of the inhalatively or systemically administered agonists of the beta-adrenergic receptor (beta-mimetics), the inhalatively administered anti-muscarinergic substances and PDE 4 inhibitors;
organic nitrates and NO donors, for example sodium nitroprusside, nitroglycerin, isosorbide mononitrate, isosorbide dinitrate, molsidomine or SIN-1, and inhaled NO;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 4 inhibitors such as roflumilast and PDE 5 inhibitors such as sildenafil, vardenafil, tadalafil, udenafil, dasantafil, avanafil, mirodenafil or lodenafil;
NO- and haem-independent activators of soluble guanylate cyclase (sGC), such as in particular the compounds described in WO 01/19355, WO 01/19776, WO 01/19778, WO 01/19780, WO 02/070462 and WO 02/070510;
NO-independent but haem-dependent stimulators of soluble guanylate cyclase (sGC), such as in particular riociguat and the compounds described in WO 00/06568, WO 00/06569, WO 02/42301, WO 03/095451, WO 2011/147809, WO 2012/004258, WO 2012/028647 and WO 2012/059549;
compounds which inhibit human neutrophil elastase (HNE), such as in particular Sivelestat, DX 890 (Reltran), and also the compounds described in WO 2004/020410, WO 2004/020412, WO 2004/024700, WO 2004/024701, WO 2005/080372, WO 2005/082863, WO 2005/082864, WO 2009/080199, WO 2009/135599, WO 2010/078953 and WO 2010/115548;
prostacyclin analogues and IP receptor agonists, by way of example and with preference iloprost, beraprost, treprostinil, epoprostenol or NS-304;
edothelin receptor antagonists, by way of example and with preference bosentan, darusentan, ambrisentan or sitaxsentan;
antiinflammatory, immunomodulating, immunosuppressive and/or cytotoxic agents, by way of example and with preference from the group of the systemically or inhalatively administered corticosteroids and also acetylcysteine, montelukast, azathioprine, cyclophosphamide, hydroxycarbamide, azithromycin, IFN-γ, pirfenidone or etanercept;
antifibrotic agents, by way of example and with preference lysophosphatidic acid receptor 1 (LPA-1) antagonists, lysyl oxidase (LOX) inhibitors, lysyl oxidase-like-2 inhibitors, vasoactive intestinal peptide (VIP), VIP analogues, αᵥβ₆-integrin antagonists, cholchicine, IFN-β, D-penicillamine, inhibitors of the WNT signal path or CCR2 antagonists;
active compounds altering lipid metabolism, for example and with preference from the group of the thyroid receptor agonists, cholesterol synthesis inhibitors such as, by way of example and preferably, HMG-CoA reductase inhibitors or squalene synthesis inhibitors, the ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists;
hypotensive active compounds, by way of example and with preference from the group of the calcium antagonists, angiotensin AII antagonists, ACE inhibitors, vasopeptidase inhibitors, endothelin antagonists, renin inhibitors, alpha receptor blockers, beta receptor blockers, mineralocorticoid receptor antagonists and also the diuretics;
compounds which inhibit the signal transduction cascade, by way of example and with preference from the group of the kinase inhibitors, in particular from the group of the tyrosine kinase and/or serine/threonine kinase inhibitors, by way of example and with preference nintedanib, dasatinib, nilotinib, bosutinib, regorafenib, sorafenib, sunitinib, cediranib, axitinib, telatinib, imatinib, brivanib, pazopanib, vatalanib, gefitinib, erlotinib, lapatinib, canertinib, lestaurtinib, pelitinib, semaxanib or tandutinib;
compounds which block the binding of serotonin to its receptors, by way of example and with preference antagonists of the 5-HT_{2B} receptor such as PRX-08066;
antagonists of growth factors, cytokines and chemokines, by way of example and with preference antagonists of TGF-β, CTGF, IL-1, IL-4, IL-5, IL-6, IL-8, IL-13 and integrins;
Rho kinase-inhibiting compounds, by way of example and with preference fasudil, Y-27632, SLx-2119, BF-66851, BF-66852, BF-66853, KI-23095 or BA-1049;
compounds which inhibit soluble epoxide hydrolase (sEH), for example N,N′-dicyclohexylurea, 12-(3-adamantan-1-ylureido)dodecanoic acid or 1-adamantan-1-yl-3-{5-[2-(2-ethoxyethoxy)ethoxy]pentyl}urea;
compounds which influence the energy metabolism of the heart, by way of example and with preference etomoxir, dichloroacetate, ranolazine or trimetazidine;
antithrombotic agents, by way of example and with preference from the group of platelet aggregation inhibitors, the anticoagulants and the profibrinolytic substances;
chemotherapeutics like those employed, for example, for the therapy of neoplasms in the lung or other organs; and/or
antibiotics, in particular from the group of fluoroquinolone carboxylic acids, such as by way of example and preferably ciprofloxacin or moxifloxacin.

In a preferred embodiment of the invention, the compounds according to the invention are administered in combination with a beta-adrenergic receptor agonist, by way of example and with preference albuterol, isoproterenol, metaproterenol, terbutalin, fenoterol, formoterol, reproterol, salbutamol or salmeterol.

In a preferred embodiment of the invention, the compounds according to the invention are administered in combination with an antimuscarinergic substance, by way of example and with preference ipratropium bromide, tiotropium bromide or oxitropium bromide.

In a preferred embodiment of the invention, the compounds according to the invention are administered in combination with a corticosteroid, by way of example and with preference prednisone, prednisolone, methylprednisolone, triamcinolone, dexamethasone, beclomethasone, betamethasone, flunisolide, budesonide or fluticasone.

Antithrombotic agents are preferably understood to mean compounds from the group of the platelet aggregation inhibitors, the anticoagulants and the profibrinolytic substances.

In a preferred embodiment of the invention, the compounds according to the invention are administered in combination with a platelet aggregation inhibitor, by way of example and with preference aspirin, clopidogrel, ticlopidin or dipyridamole.

In a preferred embodiment of the invention, the compounds according to the invention are administered in combination with a thrombin inhibitor, by way of example and with preference ximelagatran, melagatran, dabigatran, bivalirudin or clexane.

In a preferred embodiment of the invention, the compounds according to the invention are administered in combination with a GPIIb/IIIa antagonist, by way of example and with preference tirofiban or abciximab.

In a preferred embodiment of the invention, the compounds according to the invention are administered in combination with a factor Xa inhibitor, by way of example and with preference rivaroxaban, apixaban, fidexaban, razaxaban, fondaparinux, idraparinux, DU-176b, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or with a low molecular weight (LMW) heparin derivative.

In a preferred embodiment of the invention, the compounds according to the invention are administered in combination with a vitamin K antagonist, by way of example and with preference coumarin.

Hypotensive agents are preferably understood to mean compounds from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics.

In a preferred embodiment of the invention, the compounds according to the invention are administered in combination with a calcium antagonist, by way of example and with preference nifedipine, amlodipine, verapamil or diltiazem.

In a preferred embodiment of the invention, the compounds according to the invention are administered in combination with an alpha-1-receptor blocker, by way of example and with preference prazosin.

In a preferred embodiment of the invention, the compounds according to the invention are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with an angiotensin AII antagonist, by way of example and with preference losartan, candesartan, valsartan, telmisartan or embursatan.

In a preferred embodiment of the invention, the compounds according to the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds according to the invention are administered in combination with an endothelin antagonist, by way of example and with preference bosentan, darusentan, ambrisentan or sitaxsentan.

In a preferred embodiment of the invention, the compounds according to the invention are administered in combination with a renin inhibitor, by way of example and with preference aliskiren, SPP-600 or SPP-800.

In a preferred embodiment of the invention, the compounds according to the invention are administered in combination with a mineralocorticoid receptor antagonist, by way of example and with preference spironolactone, eplerenone or finerenone.

In a preferred embodiment of the invention, the compounds according to the invention are administered in combination with a diuretic, by way of example and with preference furosemide, bumetanide, torsemide, bendroflumethiazide, chlorthiazide, hydrochlorthiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or

PPAR-delta agonists, cholesterol absorption inhibitors, polymeric bile acid adsorbents, bile acid reabsorption inhibitors, lipase inhibitors and the lipoprotein(a) antagonists.

In a preferred embodiment of the invention, the compounds according to the invention are administered in combination with a CETP inhibitor, by way of example and with preference torcetrapib (CP-529 414), JJT-705 or CETP vaccine (Avant).

In a preferred embodiment of the invention, the compounds according to the invention are administered in combination with a thyroid receptor agonist, by way of example and with preference D-thyroxin, 3,5,3′-triiodothyronin (T3), CGS 23425 or axitirome (CGS 26214).

In a preferred embodiment of the invention, the compounds according to the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds according to the invention are administered in combination with a squalene synthesis inhibitor, by way of example and with preference BMS-188494 or TAK-475.

In a preferred embodiment of the invention, the compounds according to the invention are administered in combination with an ACAT inhibitor, by way of example and with preference avasimibe, melinamide, pactimibe, eflucimibe or SMP-797.

In a preferred embodiment of the invention, the compounds according to the invention are administered in combination with an MTP inhibitor, by way of example and with preference implitapide, BMS-201038, R-103757 or JTT-130.

In a preferred embodiment of the invention, the compounds according to the invention are administered in combination with a PPAR-gamma agonist, by way of example and with preference pioglitazone or rosiglitazone.

In a preferred embodiment of the invention, the compounds according to the invention are administered in combination with a PPAR-delta agonist, by way of example and with preference GW 501516 or BAY 68-5042.

In a preferred embodiment of the invention, the compounds according to the invention are administered in combination with a cholesterol absorption inhibitor, by way of example and with preference ezetimibe, tiqueside or pamaqueside.

In a preferred embodiment of the invention, the compounds according to the invention are administered in combination with a lipase inhibitor, by way of example and with preference orlistat.

In a preferred embodiment of the invention, the compounds according to the invention are administered in combination with a polymeric bile acid adsorbent, by way of example and with preference cholestyramine, colestipol, colesolvam, CholestaGel or colestimide.

In a preferred embodiment of the invention, the compounds according to the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the compounds according to the invention are administered in combination with a lipoprotein(a) antagonist, by way of example and with preference gemcabene calcium (CI-1027) or nicotinic acid.

Particular preference is given to combinations of the compounds according to the invention with one or more further active ingredients selected from the group consisting of corticosteroids, beta-adrenergic receptor agonists, anti-muscarinergic substances, PDE 4 inhibitors, PDE 5 inhibitors, sGC activators, sGC stimulators, HNE inhibitors, prostacyclin analogues, endothelin antagonists, statins, antifibrotic agents, anti-inflammatory agents, immunomodulating agents, immunosuppressive agents and cytotoxic agents.

The present invention further provides medicaments which comprise at least one compound according to the invention, typically together with one or more inert, nontoxic, pharmaceutically suitable excipients, and the use thereof for the aforementioned purposes.

The compounds according to the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds according to the invention can be administered in suitable administration forms for these administration routes.

Suitable administration forms for oral administration are those which work according to the prior art and release the compounds according to the invention rapidly and/or in a modified manner and which contain the compounds according to the invention in crystalline and/or amorphized and/or dissolved form, for example tablets (uncoated or coated tablets, for example with gastric juice-resistant or retarded-dissolution or insoluble coatings which control the release of the compound according to the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can bypass an absorption step (e.g. intravenously, intraarterially, intracardially, intraspinally or intralumbally) or include an absorption (e.g. inhalatively, intramuscularly, subcutaneously, intracutaneously, percutaneously or intraperitoneally). Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers, metered aerosols), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Preference is given to oral, intrapulmonary (inhalative) and intravenous administration.

The compounds according to the invention can be converted to the administration forms mentioned. This can be accomplished in a manner known per se by mixing with inert, non-toxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

In general, it has been found to be advantageous in the case of parenteral administration to administer amounts of from about 0.001 to 1 mg/kg, preferably about 0.01 to 0.5 mg/kg, of body weight to achieve effective results. In the case of oral administration the dosage is about 0.01 to 100 mg/kg, preferably about 0.01 to 20 mg/kg and very preferably 0.1 to 10 mg/kg of body weight. In the case of intrapulmonary administration, the amount is generally about 0.1 to 50 mg per inhalation.

It may nevertheless be necessary where appropriate to deviate from the stated amounts, specifically as a function of body weight, route of administration, individual response to the active compound, nature of the preparation and time or interval over which administration takes place. Thus, in some cases less than the abovementioned minimum amount may be sufficient, while in other cases the upper limit mentioned must be exceeded. In the case of administration of greater amounts, it may be advisable to divide them into several individual doses over the day.

The working examples which follow illustrate the invention. The invention is not restricted to the examples.

### A. EXAMPLES

### Abbreviations and Acronyms

abs. absolute
Ac acetyl
aq. aqueous, aqueous solution
br. broad (in NMR signal)
Ex. Example
Bu butyl
c Concentration
ca. circa, approximately
cat. catalytic
CI chemical ionization (in MS)
d doublet (in NMR)
d day(s)
TLC thin-layer chromatography
DCI direct chemical ionization (in MS)
dd doublet of doublets (in NMR)
DEAD diethyl azodicarboxylate
DMF N,N-dimethylformamide
DMSO dimethyl sulphoxide
Dt doublet of triplets (in NMR)
of th. of theory (chemical yield)
ee enantiomeric excess
EI electron impact ionization (in MS)
ent enantiomerically pure, enantiomer
eq. equivalent(s)
ESI electrospray ionization (in MS)
Et Ethyl
h hour(s)
HPLC high-pressure, high-performance liquid chromatography
iPr isopropyl
conc concentrated (in the case of a solution)
LC liquid chromatography
LC/MS liquid chromatography-coupled mass spectrometry
Lit. literature (reference)
m multiplet (in NMR)
Me Methyl
min minute(s)
MPLC medium-pressure liquid chromatography (on silica gel; also referred to as flash chromatography)
Ms methanesulphonyl (mesyl)
MS mass spectrometry
NMO N-methylmorpholine N-oxide
NMR nuclear magnetic resonance spectrometry
Pr propyl
q (or quart) quartet (in NMR)
qd quartet of doublets (in NMR)
quant. quantitative (in chemical yield)
quint quintet (in NMR)
rac racemic, racemate
Rf retention index (in TLC)
RP reverse phase (in HPLC)
RT room temperature
Rₜ retention time (in HPLC, LC/MS)
s singlet (in NMR)
sept septet (in NMR)
SFC supercritical liquid chromatography
t triplet (in NMR)
tBu tert-butyl
td triplet of doublets (in NMR)
TFA trifluoroacetic acid
THF tetrahydrofuran
UV ultraviolet spectrometry
v/v ratio by volume (of a solution)
tog. together

### HPLC and LC/MS Methods:

### Method 1 (LC/MS):

Instrument: Waters ACQUITY SQD UPLC system; column: Waters Acquity UPLC HSS T3 1.8μ 50×1 mm; mobile phase A: 1 l of water+0.25 ml of 99% strength formic acid; mobile phase B: 1 l of acetonitrile+0.25 ml of 99% strength formic acid; gradient: 0.0 min 90% A→1.2 min 5% A→2.0 min 5% A oven: 50° C.; flow rate: 0.40 ml/min; UV detection: 208-400 nm

### Method 2 (LC/MS):

Instrument: Micromass Quattro Premier with Waters UPLC Acquity; column: Thermo Hypersil GOLD 1.9μ, 50×1 mm; mobile phase A: 1 l of water+0.5 ml of 50% strength formic acid; mobile phase B: 1 l of acetonitrile+0.5 ml of 50% strength formic acid; gradient: 0.0 min 97% A→0.5 min 97% A→3.2 min 5% A→4.0 min 5% A; oven: 50° C.; flow rate: 0.3 ml/min; UV detection: 210 nm

### Method 3 (Preparative HPLC):

Column: Reprosil C18, 10 μm, 250×30 mm; mobile phase: acetonitrile/water with 0.1% of TFA; gradient: 0-5.00 min 10:90, sample injection at 3.00 min; 5.00-23.00 min to 95:5; 23.00-30.00 min 95:5; 30.00-30.50 min to 10:90; 30.50-31.20 min 10:90.

### Method 4 (Preparative HPLC):

Column: Reprosil C18, 10 μm, 250×30 mm; mobile phase: acetonitrile/water with 0.1% of TFA; gradient: 0-5.00 min 10:90, sample injection at 3.00 min; 5.00-20.00 min to 95:5; 20.00-30.00 min 95:5; 30.00-30.50 min to 10:90; 30.50-31.20 min 10:90.

### Single Crystal X-Ray Structural Analysis:

Single crystal: obtained by crystallization from ethanol at RT; diffractometer: Bruker diffractometer equipped with an Apex-II-CCD area detector; radiation: CuKα-radiation 1.54178 Å; temperature: 110 K; monochromator: Mirror; θ range: 5.53-67.02°; Scan type: full sphere data collection omega and phi scans; index ranges: −6≦h≦6, −38≦k≦37, −7≦1≦7; collected reflections: 21884; independent reflections: 4073 [R(int)=0.0633]; completeness to theta: 67.68° 97.8%.

Structural solution and refinement: Structural solution by direct method (SHELXS); structural refinement: least-squares refinement, hydrogen atoms in ideal positions calculated and isotropically refined; number of refined parameters: 326; final R indices (obs. Data): R1=0.0413, wR2=0.0926; R indices (all data): R1=0.0561, wR2=0.0984; data-to-parameter ratio: 12.49; quality of fit to F2: 1.019; Flack parameter: 0.02(12).

### Further Details:

The percentages in the example and test descriptions which follow are, unless indicated otherwise, percentages by weight; parts are parts by weight. Solvent ratios, dilution ratios and concentration data for the liquid/liquid solutions are in each case based on volume.

Purities are generally based on corresponding peak integrations in the LC/MS chromatogram, but they may additionally have been determined with the aid of the ¹H-NMR spectrum. If no purity is indicated, the purity is generally 100% according to automated peak integration in the LC/MS chromatogram, or the purity has not been determined explicitly.

Stated yields in % of theory are generally corrected for purity if a purity of <100% is indicated. In solvent-containing or contaminated batches, the formal yield may be “>100%”; in these cases the yield is not corrected for solvent or purity.

Some of the descriptions below of the coupling patterns of ¹H-NMR signals were taken directly from the suggestions of the ACD SpecManager (ACD/Labs Release 12.00, Product version 12.5) and have not necessarily been rigorously checked. In some cases, the suggestions of the SpecManager were adjusted manually. Manually adjusted or assigned descriptions are generally based on the optical appearance of the signals in question and do not necessarily correspond to a strict, physically correct interpretation. In general, the stated chemical shift refers to the centre of the signal in question. In the case of broad multiplets, an interval is given. Signals obscured by solvent or water were either tentatively assigned or have not been listed.

Melting points and melting-point ranges, if stated, are uncorrected.

All reactants or reagents whose preparation is not described explicitly hereinafter were purchased commercially from generally accessible sources. For all other reactants or reagents whose preparation likewise is not described hereinafter and which were not commercially obtainable or were obtained from sources which are not generally accessible, a reference is given to the published literature in which their preparation is described.

In the intermediates, illustrative examples and comparative compounds described hereinbelow, a name listed in the IUPAC pack name of the example in question “1RS,2RS,5SR” in conjunction with the statement “racemate”, is a racemic mixture of the 1R,2R,5S-enantiomer (→ in each case 1st. letter after the positional number in “1RS,2RS,5SR”) with the corresponding 1S,2S,5R-enantiomer (→ in each case 2nd. letter after the positional number). The name “1RS,2RS,5SR” in conjunction with the statements “enantiomer 1” and “enantiomer 2” means that these are the two enantiomers in separate, isolated form, where an assignment of the absolute configuration (1R,2R,5S or 1S,2S,5R) to these enantiomers has not been undertaken.

For the simplified representation of the relative stereochemical configuration of chiral centres, in the structural formulae of racemic example compounds hereinbelow only the structural formula of one of the involved enantiomers is reproduced; as is evident from the statement “racemate” for the associated IUPAC name, in these cases the second enantiomer with the opposite absolute configuration in each case is always included.

### STARTING MATERIALS AND INTERMEDIATES

### Example 1A

### 2-(Trimethylsilyl)ethyl 2-[4-(benzyloxy)phenyl]-2-hydroxybicyclo[2.2.1]heptane-7-carboxylate

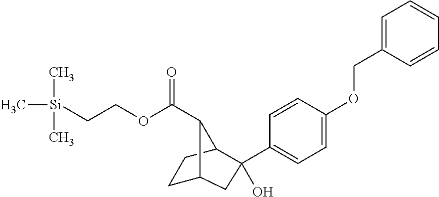 [SMILES: C[Si](C)(C)CCOC(=O)C1C2CCC1C(O)(c1ccc(OCc3ccccc3)cc1)C2]

A solution of 24.30 g (95.52 mmol) of exo-2-(trimethylsilyl)ethyl 2-oxobicyclo[2.2.1]heptane-7-carboxylate

[WO 96/15096, Example 360/Stage 1] in 60 ml of THF was slowly admixed at an internal temperature of ca. −5° C. under argon with 114.62 ml (114.62 mmol) of a 1 M solution of 4-(benzyloxy)phenylmagnesium bromide in THF, with the internal temperature rising to a maximum of 0° C. The cooling bath was then removed and the mixture was after-stirred for 1 h. The mixture was then admixed with 200 ml of 5% strength citric acid solution and extracted twice with dichloromethane. The combined organic phases were dried over magnesium sulphate and concentrated. The residue was purified by flash chromatography on 1 kg of silica gel (mobile phase cyclohexane/ethyl acetate 9:1). This gave 28.70 mg (66% of theory; purity 97%) of the title compound.

¹H-NMR (400 MHz, DMSO-d₆): δ [ppm]=7.49-7.27 (m, 7H), 6.95 (d, 2H), 5.09 (s, 2H), 5.05 (s, 1H), 4.10-4.00 (m, 2H), 2.44-2.37 (m, 1H), 2.33-2.24 (m, 1H), 2.23-2.11 (m, 1H), 1.78-1.60 (m, 1H), 1.52-1.26 (m, 4H), 0.95-0.80 (m, 2H), 0.00 (s, 9H).

LC/MS (Method 1, ESIpos): Rₜ=3.15 min; m/z=421 [M+H−H₂O]⁺

### Example 2A

### 2-(Trimethylsilyl)ethyl 2-[4-(benzyloxy)phenyl]bicyclo[2.2.1]hept-2-ene-7-carboxylate

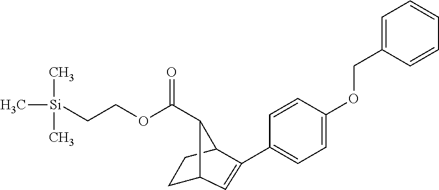 [SMILES: C[Si](C)(C)CCOC(=O)C1C2C=C(c3ccc(OCc4ccccc4)cc3)C1CC2]

### Method A:

To a solution of 28.70 g (63.466 mmol) of the compound from Example 1A in 150 ml of dichloromethane, were added under argon at ca. 0° C. firstly 26.50 ml (190.40 mmol) of triethylamine and then slowly 9.82 ml (126.93 mmol) of methanesulphonyl chloride, with the internal temperature not exceeding 5° C. The mixture was then stirred at 0° C. for 1.5 h. The mixture was then diluted with dichloromethane and extracted with water. The organic phase was dried over magnesium sulphate and concentrated and the residue was purified by flash chromatography on 1 kg of silica gel (mobile phase cyclohexane/ethyl acetate 95:5). This gave 20.06 g (75% of theory) of the title compound.

¹H-NMR (400 MHz, DMSO-d₆): δ [ppm]=7.48-7.28 (m, 7H), 6.97 (d, 2H), 6.30 (d, 1H), 5.11 (s, 2H), 4.15-4.06 (m, 2H), 3.43 (br. s, 1H), 3.06 (br. s, 1H), 1.85-1.71 (m, 2H), 1.17-1.06 (m, 1H), 1.04-0.87 (m, 3H), 0.04 (s, 9H).

LC/MS (Method 1, ESIpos): Rₜ=1.61 min; m/z=421 [M+H]⁺.

### Method B:

To a solution of 20.0 g (45.6 mmol) of the compound from Example 1A in 160 ml of pyridine were added dropwise 64.0 ml (686 mmol) of phosphorus oxychloride with stirring over a period of 10 min. The mixture was stirred for 1 h at 50° C. and then overnight at RT. The mixture was then slowly admixed with 1 litre of water and small pieces of ice, with the internal temperature being kept below 25° C. The mixture was then extracted with dichloromethane, and the combined organic phases were dried over sodium sulphate, filtered and concentrated. The residue was purified by column chromatography (silica gel, mobile phase heptane/ethyl acetate 9:1). This gave 16.3 g (85% of theory) of the title compound.

### Example 3A

### 2-(Trimethylsilyl)ethyl 2-[4-(benzyloxy)phenyl]-2,3-dihydroxybicyclo[2.2.1]heptane-7-carboxylate

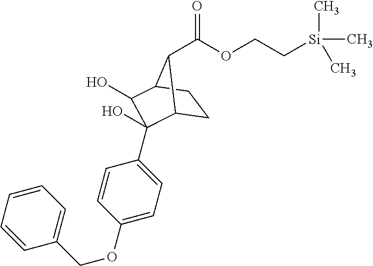 [SMILES: C[Si](C)(C)CCOC(=O)C1C2CCC1C(O)(c1ccc(OCc3ccccc3)cc1)C2O]

To a degassed solution of 25.37 g (60.314 mmol, not purity-corrected) of the compound from Example 2A in 150 ml of THF under argon was added, at 0° C., a degassed solution of 15.90 g (135.71 mmol) of N-methylmorpholine N-oxide (NMO) in 42 ml of water under argon. To this mixture were then slowly added with stirring 116 ml (9.05 mmol) of a 2.5% strength solution of osmium tetroxide in tert-butanol. The mixture was then stirred at 0° C. for 1 h. After stirring for a further 16 h at RT, the mixture was diluted with 150 ml of ethyl acetate and extracted twice with in each case 250 ml of 10% strength citric acid solution, twice with in each case 300 ml of saturated sodium hydrogen carbonate solution and twice with in each case 300 ml of saturated sodium chloride solution. The organic phase was then dried over sodium sulphate and concentrated. This gave 27.51 mg (75% of theory; purity 75%) of the title compound.

LC/MS (Method 1, ESIpos): Rₜ=1.40 min; m/z=437 421 [M+H−H₂O]⁺

### Example 4A

### 2-(Trimethylsilyl)ethyl (1RS,2RS,5SR)-2-[4-(benzyloxy)benzoyl]-5-formylcyclopentanecarboxylate (racemate)

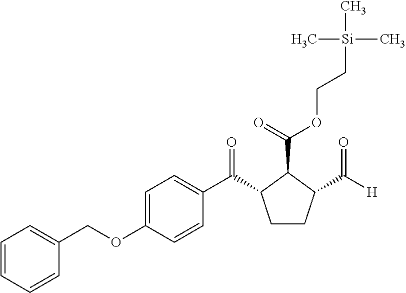 [SMILES: C[Si](C)(C)CCOC(=O)[C@H]1[C@H](C=O)CC[C@@H]1C(=O)c1ccc(OCc2ccccc2)cc1]

### Method A:

Under argon and at bath temperature of −15° C., 30.96 g (66.34 mmol, purity 95%) of lead tetraacetate were slowly added to a solution of 27.42 g (60.31 mmol, not purity-corrected) of the compound from Example 3A in 170 ml of methanol. The mixture was stirred at −15° C. for 1 h. After warming to RT, the mixture was filtered over Celite and the filtration residue was then washed three times with 50 ml of methanol in each case. The filtrate was concentrated and the residue was taken up in 500 ml of dichloromethane and 500 ml of water without phase separation being established. Thereafter, the mixture was filtered over silica gel and the silica gel was washed with dichloromethane. After phase separation, the aqueous phase was extracted again with 150 ml of dichloromethane. The combined organic phases were dried over sodium sulphate and concentrated. This gave 27.1 mg (86% of theory; purity 87%) of the title compound.

¹H-NMR (400 MHz, DMSO-d₆): δ [ppm]=9.72 (d, 1H), 8.02 (d, 2H), 7.53-7.34 (m, 5H), 7.18 (d, 2H), 5.25 (s, 2H), 4.17 (q, 1H), 4.09 (dd, 2H), 3.74 (t, 1H), 3.23-3.14 (m, 1H), 2.24-2.13 (m, 1H), 2.08-1.88 (m, 2H), 1.61-1.49 (m, 1H), 0.87-0.79 (m, 2H), 0.00 (s, 9H).

LC/MS (Method 1, ESIpos): Rₜ=1.45 min, m/z=425 [M+H−28]⁺.

### Method B:

At 0° C. and under argon, firstly 76.87 g (656 mmol) of N-methylmorpholine N-oxide (NMO) and then 2.09 g (8.20 mmol) of a 4% strength solution of osmium tetroxide in water were added to a solution of 69.0 g (131 mmol, ca. 80% purity) of the compound from Example 2A in a mixture of acetone/water/THF (3:1:1). The mixture was stirred at RT for 3 days. Then, 105.26 g (492 mmol) of sodium periodate were added and the mixture was further stirred overnight at RT. After admixing with ethyl acetate and 10% strength aqueous citric acid, the aqueous phase was separated off and extracted once with ethyl acetate. The combined organic phases were washed once with saturated sodium hydrogen carbonate solution and then stirred with magnesium silicate (Fluorisil). After filtration, the filter residue was washed with ethyl acetate. After concentrating the filtrate, the residue thus obtained was combined with the residues from two similarly carried out preliminary experiments [used amounts of the compound from Example 2A: 3.0 g (7.13 mmol) or 3.2 g (7.61 mmol)] and purified together by means of flash chromatography (silica gel, mobile phase petroleum ether/ethyl acetate 8:2). In this way, 53 g (58% of theory taking into consideration the preliminary experiments, purity 89%) of the title compound were obtained.

### Example 5A

### 2-(Trimethylsilyl)ethyl (1RS,2RS,5SR)-2-[4-(benzyloxy)benzoyl]-5-(hydroxymethyl)cyclopentanecarboxylate (racemate)

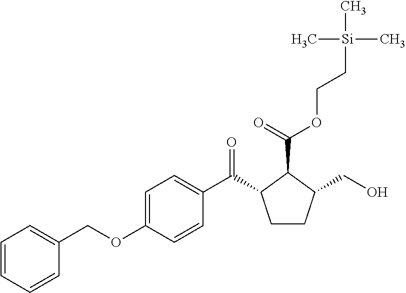 [SMILES: C[Si](C)(C)CCOC(=O)[C@H]1[C@H](CO)CC[C@@H]1C(=O)c1ccc(OCc2ccccc2)cc1]

At RT, 677 mg (17.895 mmol) of sodium borohydride were slowly added to a solution of 27.0 g (59.65 mmol, not purity-corrected) of the compound from Example 4A in 135 ml of ethanol, and the mixture was stirred at RT for 30 min. The mixture was then admixed with in each case 400 ml of ammonium chloride solution and water, and extracted twice with 300 ml of ethyl acetate in each case. The combined organic phases were dried over sodium sulphate and concentrated. This gave 21.90 mg (70% of theory; purity 87%) of the title compound.

¹H-NMR (400 MHz, DMSO-d₆): δ [ppm]=7.95 (d, 2H), 7.48-7.31 (m, 5H), 7.12 (d, 2H), 5.20 (s, 2H), 4.64 (t, 1H), 4.07-3.98 (m, 3H), 3.53-3.45 (m, 1H), 3.40-3.34 (m, 1H), 2.94 (t, 1H), 2.34-2.23 (m, 1H), 2.12-2.01 (m, 1H), 1.90-1.78 (m, 1H), 1.67-1.47 (m, 2H), 0.82-0.75 (m, 2H), 0.00 (s, 9H).

LC/MS (Method 1, ESIpos): Rₜ=1.34 min; m/z=455 [M+H]⁺.

### Example 6A

### 2-(Trimethylsilyl)ethyl (1RS,2RS,5SR)-2-[4-(benzyloxy)benzoyl]-5-[(4-oxo-1,2,3-benzotriazin-3(4H)-yl)methyl]cyclopentanecarboxylate (racemate)

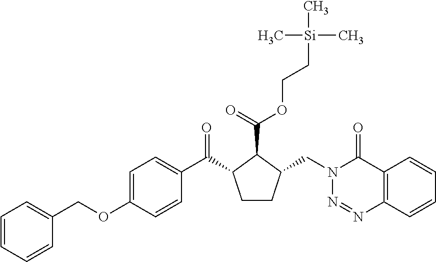 [SMILES: C[Si](C)(C)CCOC(=O)[C@H]1[C@H](Cn2nnc3ccccc3c2=O)CC[C@@H]1C(=O)c1ccc(OCc2ccccc2)cc1]

Under argon, 243 mg (1.65 mmol) of 1,2,3-benzotriazin-4(3H)-one and 1.11 g (5.50 mmol) of tributylphosphane were added to a solution of 500 mg (1.10 mmol, not purity-corrected) of the compound from Example 5A in 6 ml of THF. Then, 1.50 ml (3.30 mmol) of a 40% strength solution of diethyl azodicarboxylate (DEAD) in toluene were added dropwise at 0° C. The mixture was stirred at RT for ca. 1 h, then diluted with ethyl acetate and extracted twice with in each case 5 ml of water and twice with saturated sodium chloride solution. The organic phase was dried over magnesium sulphate and then concentrated. The residue was purified by preparative HPLC (Method 4). This gave 334 mg (52% of theory) of the title compound.

¹H-NMR (400 MHz, DMSO-d₆): δ [ppm]=8.44 (dd, 1H), 8.38 (d, 1H), 8.27 (td, 1H), 8.15-8.08 (m, 3H), 7.65-7.48 (m, 5H), 7.29 (d, 2H), 5.37 (s, 2H), 4.74-4.62 (m, 2H), 4.26 (q, 1H), 3.40 (t, 1H), 3.13-3.01 (m, 1H), 2.36-2.25 (m, 1H), 2.21-2.10 (m, 1H), 1.96-1.84 (m, 1H), 1.77-1.65 (m, 1H), 0.53-0.46 (m, 2H), 0.17 (s, 9H).

LC/MS (Method 1, ESIpos): Rₜ=1.51 min; m/z=584 [M+H]⁺.

### EMBODIMENT EXAMPLES

### Example 1

### (+/−)-(1RS,2RS,5SR)-2-[4-(Benzyloxy)benzoyl]-5-[(4-oxo-1,2,3-benzotriazin-3(4H)-yl)methyl]cyclopentanecarboxylic acid (racemate)

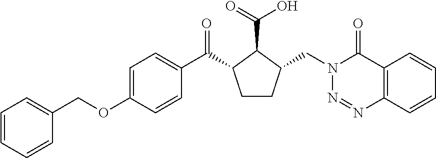 [SMILES: O=C(O)[C@H]1[C@H](Cn2nnc3ccccc3c2=O)CC[C@@H]1C(=O)c1ccc(OCc2ccccc2)cc1]

A solution of 213 mg (0.365 mmol) of the compound from Example 6A in 2 ml of dichloromethane was admixed at 0° C. with 1 ml (12.98 mmol) of trifluoroacetic acid. The mixture was stirred for 1 h at 0° C. and then stored at 5° C. for ca. 18 h. The mixture was then concentrated, the residue was taken up in dichloromethane and the solution was concentrated again. This procedure was repeated several times. Finally, the residue was taken up in acetonitrile/THF and purified by preparative HPLC (method 3). This thus gave 125 mg (71% of theory) of the title compound.

¹H-NMR (400 MHz, DMSO-d₆): δ [ppm]=12.15 (s, 1H), 8.26 (d, 1H), 8.20 (d, 1H), 8.11-8.05 (m, 1H), 8.01-7.89 (m, 3H), 7.52-7.28 (m, 5H), 7.13 (d, 2H), 5.21 (s, 2H), 4.53 (dd, 2H), 4.15-4.06 (m, 1H), 3.24 (t, 1H), 2.93-2.80 (m, 1H), 2.17-2.04 (m, 1H), 1.94-1.83 (m, 1H), 1.72-1.60 (m, 1H), 1.57-1.44 (m, 1H).

LC/MS (Method 1, ESIpos): Rₜ=1.16 min; m/z=484 [M+H]⁺.

### Separation of the Enantiomers:

### Method A:

645 mg of the racemic compound from Example 1 were dissolved in 20 ml of dioxane and separated into the enantiomers by preparative HPLC on a chiral phase (see Examples 2 and 3) [column: Daicel Chiralpak IC, 5 μm 250 mm×20 mm; flow rate: 15 ml/min; detection: 220 nm; injection volume: 0.2 ml; temperature: 25° C.; mobile phase: t=0-5 min 80% methanol/20% acetonitrile].

### Method B:

510 mg of the racemic compound from Example 1 were dissolved in 10 ml of THF at elevated temperature and separated into the enantiomers by preparative SFC on a chiral phase (see Example 2 and 3) [column: Daicel Chiralpak AS-H, 5 μm, 250 mm×20 mm; flow rate: 100 ml/min; detection: 210 nm; injection volume: 0.25 ml; temperature: 40° C.; mobile phase: t=0-8 min 60% carbon dioxide/40% ethanol].

### Example 2

### (+)-(1S,2S,5R)-2-[4-(Benzyloxy)benzoyl]-5-[(4-oxo-1,2,3-benzotriazin-3(4H)-yl)methyl]cyclopentanecarboxylic acid

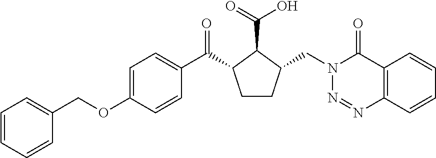 [SMILES: O=C(O)[C@H]1[C@H](Cn2nnc3ccccc3c2=O)CC[C@@H]1C(=O)c1ccc(OCc2ccccc2)cc1]

Yield (according to Method A): 209 mg; ee-value=99%

[α]_{D}²⁰=+67.2°, 589 nm, c=0.32 g/100 ml, chloroform

¹H-NMR (400 MHz, DMSO-d₆): δ [ppm]=12.15 (s, 1H), 8.26 (d, 1H), 8.20 (d, 1H), 8.11-8.05 (m, 1H), 8.01-7.90 (m, 3H), 7.49-7.31 (m, 5H), 7.13 (d, 2H), 5.21 (s, 2H), 4.53 (dd, 2H), 4.15-4.06 (m, 1H), 3.24 (t, 1H), 2.94-2.80 (m, 1H), 2.17-2.03 (m, 1H), 1.94-1.82 (m, 1H), 1.72-1.60 (m, 1H), 1.57-1.44 (m, 1H).

LC/MS (Method 2, ESIpos): Rₜ=2.59 min; m/z=484 [M+H]⁺.

A single-crystal X-ray structural analysis produced a (1S,2S,5R)-absolute configuration for this enantiomer. The resulting crystal data are shown in the table below (for the description of the method see introductory paragraph of the experimental section).

Crystal Data from X-Ray Structural Analysis for Example 2:

| Space group | P21 |
|---|---|
| Cell structure | |
| a (Å) | 5.7051(3) |
| b (Å) | 31.9892(14) |
| c (Å) | 6.3511(3) |
| α (°) | 90 |
| β (°) | 94.405(3) |
| γ (°) | 90 |
| Volume (Å³) | 1155.66(10) |
| molecules per unit cell | 2 |
| calculated density (Mg/m³) | 1.389 |

### Example 3

### (−)-(1R,2R,5S)-2-[4-(Benzyloxy)benzoyl]-5-[(4-oxo-1,2,3-benzotriazin-3(4H)-yl)methyl]cyclopentanecarboxylic acid

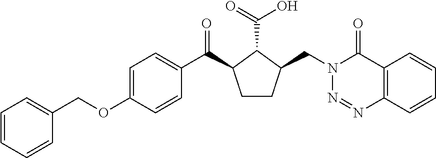 [SMILES: O=C(O)[C@@H]1[C@@H](Cn2nnc3ccccc3c2=O)CC[C@H]1C(=O)c1ccc(OCc2ccccc2)cc1]

Yield (according to method A): 228 mg; ee value=99%

[α]_{D}²⁰=−68.3°, 589 nm, c=0.35 g/100 ml, chloroform

¹H-NMR (400 MHz, DMSO-d₆): δ [ppm]=12.15 (s, 1H), 8.26 (d, 1H), 8.20 (d, 1H), 8.11-8.05 (m, 1H), 8.01-7.89 (m, 3H), 7.49-7.31 (m, 5H), 7.13 (d, 2H), 5.21 (s, 2H), 4.53 (dd, 2H), 4.14-4.05 (m, 1H), 3.24 (t, 1H), 2.94-2.80 (m, 1H), 2.17-2.04 (m, 1H), 1.95-1.83 (m, 1H), 1.72-1.60 (m, 1H), 1.57-1.44 (m, 1H).

LC/MS (Method 2, ESIpos): Rₜ=2.59 min; m/z=484 [M+H]⁺.

### COMPARATIVE EXAMPLES

### Comparative Example A-1

### (1RS,2RS,5SR)-2-[(4′-chlorobiphenyl-4-yl)carbonyl]-5-[(4-oxo-1,2,3-benzotriazin-3(4H)-yl)methyl]cyclopentanecarboxylic acid (racemate)

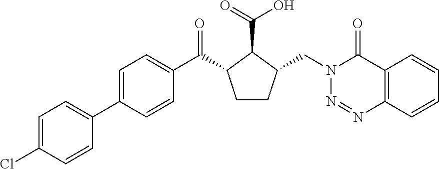 [SMILES: O=C(O)[C@H]1[C@H](Cn2nnc3ccccc3c2=O)CC[C@@H]1C(=O)c1ccc(-c2ccc(Cl)cc2)cc1]

The racemic compound and its preparation is described in WO 97/43239-A1 as Example 1.

### Separation of the Enantiomers:

1.450 g (2.97 mmol) of (1RS,2RS,5SR)-2-[(4′-chlorobiphenyl-4-yl)carbonyl]-5-[(4-oxo-1,2,3-benzotriazin-3(4H)-yl)methyl]cyclopentanecarboxylic acid (racemate) were dissolved in a mixture of 80 ml of ethanol and 20 ml of acetonitrile and separated into the enantiomers by preparative HPLC on a chiral phase (see Comparative Examples A-2 and A-3) [column: Daicel Chiralpak ID 5 μm 250 mm×20 mm; flow rate: 12 ml/min; detection: 220 nm; injection volume: 1.8 ml; temperature: 45° C.; mobile phase: 100% ethanol isocratic; run time: 12 min]:

### Comparative Example A-2

### (1RS,2RS,5SR)-2-[(4′-chlorobiphenyl-4-yl)carbonyl]-5-[(4-oxo-1,2,3-benzotriazin-3(4H)-yl)methyl]cyclopentanecarboxylic acid (enantiomer 1)

This gave 637 mg (chemical purity 100%) of the title compound.

Rₜ=5.59 min, ee value=99% [column: Daicel Chiralpak IC-H 250 mm×4.6 mm, 5 μm; flow rate: 1.0 ml/min; detection: 220 nm; temperature: 45° C.; mobile phase: 100% ethanol+0.2% TFA+1% water, isocratic].

### Comparative Example A-3

### (1RS,2RS,5SR)-2-[(4′-Chlorobiphenyl-4-yl)carbonyl]-5-[(4-oxo-1,2,3-benzotriazin-3(4H)-yl)methyl]cyclopentanecarboxylic acid (enantiomer 2)

This gave 651 mg (chemical purity 100%) of the title compound.

Rₜ=8.51 min, ee value=99% [column: Daicel Chiralpak IC-H 250 mm×4.6 mm, 5 μm; flow rate: 1.0 ml/min; detection: 220 nm; temperature: 45° C.; mobile phase: 100% ethanol+0.2% TFA+1% water, isocratic].

### Comparative Example B-1

### (+/−)-4-Oxo-2-[2-(4-oxo-1,2,3-benzotriazin-3(4H)-yl)ethyl]-4-[4-(pentyloxy)phenyl]butanoic acid (racemate)

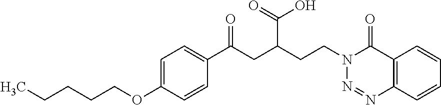 [SMILES: CCCCCOc1ccc(C(=O)CC(CCn2nnc3ccccc3c2=O)C(=O)O)cc1]

The racemic compound and its preparation is described in WO 97/43237-A1 as Example 15.

### Separation of the Enantiomers:

250 mg (0.57 mmol) of (+/−)-4-oxo-2-[2-(4-oxo-1,2,3-benzotriazin-3(4H)-yl)ethyl]-4-[4-(pentyloxy)phenyl]butanoic acid (racemate) were dissolved in 7 ml of acetonitrile and separated into the enantiomers by preparative HPLC on a chiral phase (see Comparative Examples B-2 and B-3) [column: Daicel Chiralpak AD-H, 5 μm, 250 mm×20 mm; flow rate: 20 ml/min; detection: 280 nm; injection volume: 0.12 ml; temperature: 25° C.; mobile phase: 80% acetonitrile/20% ethanol+0.2% glacial acetic acid, isocratic; run time: 6 min]:

### Comparative Example B-2

### (+)-4-Oxo-2-[2-(4-oxo-1,2,3-benzotriazin-3(4H)-yl)ethyl]-4-[4-(pentyloxy)phenyl]butanoic acid (enantiomer 1)

This gave 111 mg (chemical purity 100%) of the title compound.

[α]_{D}²⁰=+30.6°, 589 nm, c=0.32 g/100 ml, chloroform

Rₜ=8.21 min, ee value=100% [column: Daicel Chiralpak AD-H, 250 mm×4.6 mm, 5 μm; flow rate: 1.0 ml/min; detection: 280 nm; mobile phase: 80% acetonitrile+0.2% glacial acetic acid/20% ethanol+0.2% glacial acetic acid, isocratic].

### Comparative Example B-3

### (−)-4-Oxo-2-[2-(4-oxo-1,2,3-benzotriazin-3(4H)-yl)ethyl]-4-[4-(pentyloxy)phenyl]butanoic acid (enantiomer 2)

This gave 119 mg (chemical purity 100%) of the title compound.

[α]_{D}²⁰=−25.6°, 589 nm, c=0.35 g/100 ml, chloroform

Rₜ=10.34 min, ee value=99% [column: Daicel Chiralpak AD-H, 250 mm×4.6 mm, 5 μm; flow rate: 1.0 ml/min; detection: 280 nm; mobile phase: 80% acetonitrile+0.2% glacial acetic acid/20% ethanol+0.2% glacial acetic acid, isocratic].

### B. ASSESSMENT OF PHARMACOLOGICAL EFFICACY

The pharmacological activity of the compounds according to the invention can be demonstrated by in vitro and in vivo studies, as known to the person skilled in the art. The application examples which follow describe the biological action of the compounds according to the invention, without restricting the invention to these examples.

### ABBREVIATIONS AND ACRONYMS

APMA 4-aminophenyl mercury acetate
Brij®-35 polyoxyethylene lauryl ether
BSA bovine serum albumin
CYP cytochrome P450
Dap (or Dpa) L-2,3-diaminopropionic acid (β-amino-1-alanine)
DMSO dimethyl sulphoxide
Dnp 2,4-dinitrophenyl
EDTA ethylenediaminetetraacetic acid
HEPES 2-[4-(2-Hydroxyethyl)piperazin-1-yl]ethanesulphonic acid
HME human macrophage elastase
IC inhibition concentration
i.v. intravenous
Mca (7-methoxycoumarin-4-yl)acetyl
MMP matrix metallopeptidase
MTP microtiter plate
NADP⁺ nicotinamide adenine dinucleotide phosphate (oxidized form)
NADPH nicotinamide adenine dinucleotide phosphate (reduced form)
Nval norvalin
PEG polyethylene glycol
p.o. peroral
Tris tris(hydroxymethyl)aminomethane
v/v ratio by volume (of a solution)
w/w ratio by weight (of a solution)

### B-1. In Vitro HME Inhibition Test

The activity of the compounds according to the invention towards HME (MMP-12) is ascertained in an in vitro inhibition test. The HME-mediated amidolytic cleavage of a suitable peptide substrate leads herein to a fluorescent light increase. The signal intensity of the fluorescent light is directly proportional to the enzyme activity. The active concentration of a test compound at which half of the enzyme is inhibited (50% signal intensity of the fluorescent light) is given as IC₅₀ value.

### Standard In Vitro HME Inhibition Test:

In a 384 hole microtiter plate, in a test volume of in total 41 μl of the test buffer (0.1 M HEPES pH 7.4, 0.15 M NaCl, 0.03 M CaCl₂, 0.004 mM ZnCl₂, 0.02 M EDTA, 0.005% Brij®), the enzyme (0.5 nM HME; R&D Systems, 917-MP, autocatalytic activation according to the manufacturer's instructions) and the intramolecularly quenched substrate [5 μM Mca-Pro-Leu-Gly-Leu-Glu-Glu-Ala-Dap(Dnp)-NH₂; Bachem, M-2670] are incubated in the absence and presence of the test substance (as solution in DMSO) for two hours at 37° C. The fluorescent light intensity of the test batches is measured (excitation 323 nm, emission 393 nm). The IC₅₀ values are ascertained by plotting the fluorescent light intensity against the active ingredient concentration.

### High-Sensitivity In Vitro HME Inhibition Test:

If subnanomolar IC values are produced for high potent test substances in the standard HME inhibition test described above, then a modified test is used for their more precise determination. Here, a ten-fold lower enzyme concentration is used (final concentration e.g. 0.05 nM), in order to achieve an increased sensitivity of the test. The incubation time of the test is accordingly chosen to be longer (e.g. 16 hours).

### In Vitro HME Inhibition Test in the Presence of Serum Albumin in the Reaction Buffer:

This test corresponds to the standard HME inhibition test described above, but using a modified reaction buffer. This reaction buffer additionally comprises bovine serum albumin (BSA, fatty acid-free, A6003, Sigma-Aldrich) of a final concentration of 2% (w/w), which corresponds to approximately half of the physiological serum albumin content. The enzyme concentration in this modified test is slightly increased (e.g. 0.75 nM), as is the incubation time (e.g. three hours).

Table 1 below gives the IC₅₀ values from these HME inhibition tests for the working examples of the present invention and also for two structurally related comparison compounds from the prior art (as racemate or separated enantiomers)→(sometimes as average values from several independent individual determinations and rounded to two significant places). The IC₅₀ values were determined for racemates and enantiomers from differently generated DMSO stock solutions. Whereas an automatically created DMSO stock solution from the internal substance logistics was used for racemates by means of a standard method, for enantiomers and for a more precise direct comparison of the enantiomers with one another, in each case a freshly produced, manually prepared DMSO stock solution was used.

TABLE 1
Inhibition of human macrophage elastase (HME/hMMP-12) in the
absence (−) or presence (+) of serum albumin (BSA)

| Example | HME IC₅₀ [nM] | HME IC₅₀ [nM] |
|---|---|---|
| No. | (−BSA) | (+BSA) |
| 1 | 0.059 | n.d. |
| 2 | 0.071 | 8.4 |
| 3 | 14 | n.d. |
| A-1 | 0.043 | n.d. |
| A-2 | 66 | n.d. |
| A-3 | 0.018 | 5.4 |
| B-1 | 1.5 | n.d. |
| B-2 | 1.6 | 170 |
| B-3 | 160 | n.d. |

[n.d. = not determined].

As is evident from the data in Table 1, the compounds 1 to 3 according to the invention are significantly more potent compared to the relevant comparison compounds A-1 to A-3 or B-1 to B-3 (more than one order of magnitude: cf. Example 1 to B-1, Example 2 to B-2, Example 3 to B-3) or are comparably potent (same order of magnitude: cf. Example 1 to A-1, Example 2 to A-3, Example 3 to A-2). A similar picture also arises under the test conditions of a potentially competing nonspecific protein binding of the compounds according to the invention and the comparison compounds, such as for example to serum albumins (IC₅₀ values in the presence of BSA: cf. Example 2 to A-3 or B-2).

Moreover, Tables 2A/2B and 3A/3B reveal a significantly higher selectivity of the compounds according to the invention compared to the relevant comparison compounds, in particular compared to those with a comparable HME activity (see therein).

### B-2. In Vitro MMP Inhibition Tests

The activity strength of the compounds according to the invention towards other MMPs (and therefore their selectivity) is likewise ascertained in in vitro inhibition tests. The MMP-mediated amidolytic cleavage of a suitable peptide substrate also leads here to a fluorescent light increase. The signal intensity of the fluorescent light is directly proportional to the enzyme activity. The active concentration of a test compound at which half of the enzyme is inhibited (50% signal intensity of the fluorescent light) is given as IC₅₀ value.

### a) Human MMPs:

### In Vitro MMP-1 Inhibition Test:

Recombinant MMP-1 (R&D Systems, 901-MP) is chemically activated in accordance with the manufacturer's instructions by using APMA. 1 μl of the test compound to be analysed (as a solution in DMSO, suitable concentrations e.g. 1 nM to 30 μM) is pipetted into 24 μl of activated enzyme (final concentration e.g. 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Pro-Leu-Gly-Leu-Dpa(Dnp)-Ala-Arg-NH₂ (final concentration e.g. 10 μM; R&D Systems, ES-001), such that a total test volume of 50 μl results. The progress of the MMP-1 reaction is measured by measuring the fluorescence intensity (excitation 320 nm, emission 410 nm) over a suitable period (e.g. over 120 min at a temperature of 32° C.).

### In Vitro MMP-2 Inhibition Test:

Recombinant MMP-2 (R&D Systems, 902-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Pro-Leu-Gly-Leu-Dpa(Dnp)-Ala-Arg-NH₂ (final concentration e.g. 10 μM; R&D Systems, ES-001), such that a total test volume of 50 μl results. The progress of the MMP-2 reaction is measured by measuring the fluorescence intensity (excitation 320 nm, emission 410 nm) over a suitable period (e.g. over 120 min at a temperature of 32° C.).

### In Vitro MMP-3 Inhibition Test:

Recombinant MMP-3 (R&D Systems, 513-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Arg-Pro-Lys-Pro-Val-Glu-Nval-Trp-Arg-Lys(Dnp)-NH₂ (final concentration e.g. 10 μM; R&D Systems, ES-002), such that a total test volume of 50 μl results. The progress of the MMP-3 reaction is measured by measuring the fluorescence intensity (excitation 320 nm, emission 410 nm) over a suitable period (e.g. over 120 min at a temperature of 32° C.).

### In Vitro MMP-7 Inhibition Test:

Recombinant MMP-7 (R&D Systems, 907-MP) is chemically activated in accordance with the manufacturer's instructions using APMA. 1 μl of the test compound to be analysed (as a solution in DMSO, suitable concentrations e.g. 1 nM to 30 μM) is pipette into 24 μl of activated enzyme (final concentration e.g. 0.5 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Pro-Leu-Gly-Leu-Dpa(Dnp)-Ala-Arg-NH₂ (final concentration e.g. 10 μM; R&D Systems, ES-001), such that a total test volume of 50 μl results. The progress of the MMP-7 reaction is measured by measuring the fluorescence intensity (excitation 320 nm, emission 410 nm) over a suitable period (e.g. over 120 min at a temperature of 32° C.).

### In Vitro MMP-8 Inhibition Test:

Recombinant MMP-8 (R&D Systems, 908-MP) is chemically activated in accordance with the manufacturer's instructions using APMA. 1 μl of the test compound to be analysed (as a solution in DMSO, suitable concentrations e.g. 1 nM to 30 μM) is pipette into 24 μl of activated enzyme (final concentration e.g. 0.5 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Pro-Leu-Gly-Leu-Dpa(Dnp)-Ala-Arg-NH₂ (final concentration e.g. 10 μM; R&D Systems, ES-001), such that a total test volume of 50 μl results. The progress of the MMP-8 reaction is measured by measuring the fluorescence intensity (excitation 320 nm, emission 410 nm) over a suitable period (e.g. over 120 min at a temperature of 32° C.).

### In Vitro MMP-9 Inhibition Test:

Recombinant MMP-9 (R&D Systems, 911-MP) is chemically activated in accordance with the manufacturer's instructions using APMA. 1 μl of the test compound to be analysed (as a solution in DMSO, suitable concentrations e.g. 1 nM to 30 μM) is pipette into 24 μl of activated enzyme (final concentration e.g. 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Pro-Leu-Gly-Leu-Dpa(Dnp)-Ala-Arg-NH₂ (final concentration e.g. 10 μM; R&D Systems, ES-001), such that a total test volume of 50 μl results. The progress of the MMP-9 reaction is measured by measuring the fluorescence intensity (excitation 320 nm, emission 410 nm) over a suitable period (e.g. over 120 min at a temperature of 32° C.).

### In Vitro MMP-10 Inhibition Test:

Recombinant MMP-10 (R&D Systems, 910-MP) is chemically activated in accordance with the manufacturer's instructions using APMA. 1 μl of the test compound to be analysed (as a solution in DMSO, suitable concentrations e.g. 1 nM to 30 μM) is pipette into 24 μl of activated enzyme (final concentration e.g. 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Arg-Pro-Lys-Pro-Val-Glu-Nval-Trp-Arg-Lys(Dnp)-NH₂ (final concentration e.g. 10 μM; R&D Systems, ES-002), such that a total test volume of 50 μl results. The progress of the MMP-10 reaction is measured by measuring the fluorescence intensity (excitation 320 nm, emission 410 nm) over a suitable period (e.g. over 120 min at a temperature of 32° C.).

### In Vitro MMP-13 Inhibition Test:

Recombinant MMP-13 (R&D Systems, 511-MP) is chemically activated in accordance with the manufacturer's instructions using APMA. 1 μl of the test compound to be analysed (as a solution in DMSO, suitable concentrations e.g. 1 nM to 30 μM) is pipette into 24 μl of activated enzyme (final concentration e.g. 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Pro-Leu-Gly-Leu-Dpa(Dnp)-Ala-Arg-NH₂ (final concentration e.g. 10 μM; R&D Systems, ES-001), such that a total test volume of 50 μl results. The progress of the MMP-13 reaction is measured by measuring the fluorescence intensity (excitation 320 nm, emission 410 nm) over a suitable period (e.g. over 120 min at a temperature of 32° C.).

### In Vitro MMP-14 Inhibition Test:

Recombinant MMP-14 (R&D Systems, 918-MP) is enzymatically activated in accordance with the manufacturer's instructions using recombinant furin (R&D Systems, 1503-SE). 1 μl of the test compound to be analysed (as a solution in DMSO, suitable concentrations e.g. 1 nM to 30 μM) is pipette into 24 μl of activated enzyme (final concentration e.g. 0.5 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Lys-Pro-Leu-Gly-Leu-Dpa(Dnp)-Ala-Arg-NH₂ (final concentration e.g. 5 μM; R&D Systems, ES-010), such that a total test volume of 50 μl results. The progress of the MMP-14 reaction is measured by measuring the fluorescence intensity (excitation 320 nm, emission 410 nm) over a suitable period (e.g. over 120 min at a temperature of 32° C.).

### In Vitro MMP-16 Inhibition Test:

Recombinant MMP-16 (R&D Systems, 1785-MP) is enzymatically activated in accordance with the manufacturer's instructions using recombinant furin (R&D Systems, 1503-SE). 1 μl of the test compound to be analysed (as a solution in DMSO, suitable concentrations e.g. 1 nM to 30 μM) is pipette into 24 μl of activated enzyme (final concentration e.g. 1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Lys-Pro-Leu-Gly-Leu-Dpa(Dnp)-Ala-Arg-NH₂ (final concentration e.g. 5 μM; R&D Systems, ES-010), such that a total test volume of 50 μl results. The progress of the MMP-16 reaction is measured by measuring the fluorescence intensity (excitation 320 nm, emission 410 nm) over a suitable period (e.g. over 120 min at a temperature of 32° C.).

Tables 2A and 2B below give the IC₅₀ values from these tests relating to the inhibition of human MMPs for representative embodiment examples of the present invention, and also for two structurally related comparison compounds from the prior art (as racemate or separated enantiomer)→(sometimes as average values from several independent individual determinations and rounded to two significant places). The IC₅₀ values were determined for racemates and enantiomers from differently generated DMSO stock solutions. Whereas an automatically produced DMSO stock solution from the internal substance logistics was used for racemates by means of a standard method, in the case of enantiomers a freshly produced, manually prepared DMSO stock solution was used in each case for a more precise direct comparison of the enantiomers with one another.

TABLE 2A
Inhibition of human MMPs

| | MMP-1 | MMP-2 | MMP-3 | MMP-7 | MMP-8 |
|---|---|---|---|---|---|
| Example | IC₅₀ | IC₅₀ | IC₅₀ | IC₅₀ | IC₅₀ |
| No. | [nM] | [nM] | [nM] | [nM] | [nM] |
| 1 | 12000 | 18 | 390 | 500 | 9.2 |
| 2 | 5800 | 11.8 | 175 | 420 | 10.1 |
| A-1 | 220 | 1.1 | 49 | 310 | <0.61 |
| A-3 | 135 | 0.70 | 22.5 | 145 | <0.61 |
| B-1 | >40000 | 450 | 9150 | >40000 | 706 |
| B-2 | >40000 | 120 | 6600 | 22500 | 275 |

TABLE 2B
Inhibition of human MMPs

| | MMP-9 | MMP-10 | MMP-13 | MMP-14 | MMP-16 |
|---|---|---|---|---|---|
| Example | IC₅₀ | IC₅₀ | IC₅₀ | IC₅₀ | IC₅₀ |
| No. | [nM] | [nM] | [nM] | [nM] | [nM] |
| 1 | 30 | 21 | 30 | 27 | 130 |
| 2 | 23 | 17.5 | 14.5 | 15 | 78.5 |
| A-1 | 1.9 | 4.6 | 1.8 | 2.9 | 16 |
| A-3 | 1.1 | <0.61 | 0.82 | 1.2 | 5.2 |
| B-1 | 1400 | 5700 | 1800 | 3800 | 22000 |
| B-2 | 625 | 1900 | 360 | 1300 | 6700 |

A comparison of the inhibition data given in Tables 1 and 2A/2B reveals that the compounds according to the invention—in particular the more active enantiomer—have a very high inhibitory potency (in the two-position picomolar range) towards HME and at the same time a very high selectivity (two to four orders of magnitude or even more) towards related human MMPs.

As is moreover evident from the data in the Tables 2A/2B, the compounds according to the invention have a significantly greater selectivity (as a rule more than one order of magnitude) or a comparable selectivity (as a rule same order of magnitude) compared to the relevant comparison compounds A-1/A-3 or B-1/B-2.

Viewed overall, it is evident from this data that the compounds according to the invention are significantly more selective compared to the relevant comparison compounds or, for a comparable selectivity, are significantly more potent, i.e. have a considerably improved profile as regards the combination of activity strength and selectivity.

### b) MMPs of Rodents:

### In Vitro MMP-2 Inhibition Test of the Mouse:

Recombinant MMP-2 of the mouse (R&D Systems, 924-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Pro-Leu-Gly-Leu-Dpa(Dnp)-Ala-Arg-NH₂ (final concentration e.g. 10 μM; R&D Systems, ES-001), such that a total test volume of 50 μl results. The progress of the MMP-2 reaction is measured by measuring the fluorescence intensity (excitation 320 nm, emission 410 nm) over a suitable period (e.g. over 120 min at a temperature of 32° C.).

### In Vitro MMP-3 Inhibition Test of the Mouse:

Recombinant MMP-3 of the mouse (R&D Systems, 548-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 0.5 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Arg-Pro-Lys-Pro-Val-Glu-Nval-Trp-Arg-Lys(Dnp)-NH₂ (final concentration e.g. 5 μM; R&D Systems, ES-002), such that a total test volume of 50 μl results. The progress of the MMP-3 reaction is measured by measuring the fluorescence intensity (excitation 320 nm, emission 410 nm) over a suitable period (e.g. over 120 min at a temperature of 32° C.).

### In Vitro MMP-7 Inhibition Test of the Mouse:

Recombinant MMP-7 of the mouse (R&D Systems, 2967-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 0.5 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Lys-Pro-Leu-Gly-Leu-Dpa(Dnp)-Ala-Arg-NH₂ (final concentration e.g. 5 μM; R&D Systems, ES-010), such that a total test volume of 50 μl results. The progress of the MMP-7 reaction is measured by measuring the fluorescence intensity (excitation 320 nm, emission 410 nm) over a suitable period (e.g. over 120 min at a temperature of 32° C.).

### In Vitro MMP-8 Inhibition Test of the Mouse:

Recombinant MMP-8 of the mouse (R&D Systems, 2904-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Lys-Pro-Leu-Gly-Leu-Dpa(Dnp)-Ala-Arg-NH₂ (final concentration e.g. 5 μM; R&D Systems, ES-010), such that a total test volume of 50 μl results. The progress of the MMP-8 reaction is measured by measuring the fluorescence intensity (excitation 320 nm, emission 410 nm) over a suitable period (e.g. over 120 min at a temperature of 32° C.).

### In Vitro MMP-9 Inhibition Test of the Mouse:

Recombinant MMP-9 of the mouse (R&D Systems, 909-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Pro-Leu-Gly-Leu-Dpa(Dnp)-Ala-Arg-NH₂ (final concentration e.g. 5 μM; R&D Systems, ES-001), such that a total test volume of 50 μl results. The progress of the MMP-9 reaction is measured by measuring the fluorescence intensity (excitation 320 nm, emission 410 nm) over a suitable period (e.g. over 120 min at a temperature of 32° C.).

### In Vitro MMP-12 Inhibition Test of the Mouse:

Recombinant MMP-12 of the mouse (R&D Systems, 3467-MP) is autocatalytically activated in accordance with the manufacturer's instructions. 1 μl of the test compound to be analysed (as a solution in DMSO, suitable concentrations e.g. 1 nM to 30 μM) is pipetted into 24 μl of activated enzyme (final concentration e.g. 1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Lys-Pro-Leu-Gly-Leu-Dpa(Dnp)-Ala-Arg-NH₂ (final concentration e.g. 5 μM; R&D Systems, ES-010), such that a total test volume of 50 μl results. The progress of the MMP-12 reaction is measured by measuring the fluorescence intensity (excitation 320 nm, emission 410 nm) over a suitable period (e.g. over 120 min at a temperature of 32° C.).

### High-Sensitivity In Vitro MMP-12 Inhibition Test of the Mouse:

If subnanomolar IC values are produced for high-potency test substances in the above-described MMP-12 inhibition test of the mouse, then a modified test is used for their more precise determination. Here, a ten-fold lower enzyme concentration is used (final concentration e.g. 0.1 nM), in order to achieve an increased sensitivity of the test. The incubation time of the test is correspondingly chosen to be longer (e.g. 16 hours).

### In Vitro MMP-2 Inhibition Test of the Rat:

Recombinant MMP-2 of the rat (R&D Systems, 924-MP) is chemically activated in accordance with the manufacturer's instructions using APMA. 0.1 μl of the test compound to be analysed (as a solution in DMSO, suitable concentrations e.g. 1 nM to 30 μM) is pipetted into 24 μl of activated enzyme (final concentration e.g. 1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Pro-Leu-Gly-Leu-Dpa(Dnp)-Ala-Arg-NH₂ (final concentration e.g. 10 μM; R&D Systems, ES-001), such that a total test volume of 50 μl results. The progress of the MMP-2 reaction is measured by measuring the fluorescence intensity (excitation 320 nm, emission 410 nm) over a suitable period (e.g. over 120 min at a temperature of 32° C.).

### In Vitro MMP-8 Inhibition Test of the Rat:

Recombinant MMP-8 of the rat (R&D Systems, 3245-MP) is chemically activated in accordance with the manufacturer's instructions using APMA. 2 μl of the test compound to be analysed (as a solution in DMSO, suitable concentrations e.g. 1 nM to 30 μM) is pipetted into 24 μl of activated enzyme (final concentration e.g. 1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Lys-Pro-Leu-Gly-Leu-Dpa(Dnp)-Ala-Arg-NH₂ (final concentration e.g. 5 μM; R&D Systems, ES-010), such that a total test volume of 50 μl results. The progress of the MMP-8 reaction is measured by measuring the fluorescence intensity (excitation 320 nm, emission 410 nm) over a suitable period (e.g. over 120 min at a temperature of 32° C.).

### In Vitro MMP-9 Inhibition Test of the Rat:

Recombinant MMP-9 of the mouse (R&D Systems, 5427-MM) is chemically activated in accordance with the manufacturer's instructions using APMA. 0.1 μl of the test compound to be analysed (as a solution in DMSO, suitable concentrations e.g. 1 nM to 30 μM) is pipetted into 24 μl of activated enzyme (final concentration e.g. 1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Pro-Leu-Gly-Leu-Dpa(Dnp)-Ala-Arg-NH₂ (final concentration e.g. 5 μM; R&D Systems, ES-001), such that a total test volume of 50 μl results. The progress of the MMP-9 reaction is measured by measuring the fluorescence intensity (excitation 320 nm, emission 410 nm) over a suitable period (e.g. over 120 min at a temperature of 32° C.).

### In Vitro MMP-12 Inhibition Test of the Rat:

MMP-12 of the rat (Uniprot NP_446415.1; construct L96-V277) is expressed with an additional N-terminal His-Tag and a consecutive TEV cleavage sequence by means of a pDEco7 vector in E. coli (BL21). The thus recombinantly expressed protein forms an intracellular insoluble protein compartment (so-called inclusion body). This is solubilized after separation and intensive washing under denaturing conditions. For this, the inclusion body pellet fraction from a 250 ml E. coli culture is taken up in a volume of 120 ml of buffer A (50 mM Tris pH 7.4, 100 mM NaCl, 0.03 mM ZnCl₂, 10 mM CaCl₂, 8 M urea). The soluble protein is renatured by in each case dialysing 60 ml of the sample several times at 4-8° C. against buffer B (50 mM Tris pH 7.4, 100 mM NaCl, 0.03 mM ZnCl₂, 10 mM CaCl₂). After the dialysis, the sample is centrifuged (25 000×g). The folded-back protein is obtained in the supernatant with a yield of 3.7 mg per 250 ml E. coli culture. The thus obtained protein is enzymatically active without further purification operations or protease-mediated cleavage processes.

1 μl of the test compound to be analysed (as a solution in DMSO, suitable concentrations e.g. 1 nM to 30 μM) is pipetted into 24 μl of MMP-12 protein (final concentration e.g. 1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched substrate Mca-Pro-Leu-Gly-Leu-Dpa(Dnp)-Ala-Arg-NH₂ (final concentration e.g. 5 μM; R&D Systems, ES-001), such that a total test volume of 50 μl results. The progress of the MMP-12 reaction is measured by measuring the fluorescence intensity (excitation 320 nm, emission 410 nm) over a suitable period (e.g. over 120 min at a temperature of 32° C.).

Tables 3A and 3B below give the IC₅₀ values from these tests for the inhibition of rodent MMPs for representative embodiment examples of the present invention and also for two structurally related comparison compounds from the prior art (as racemate and separated enantiomer)→(in part as average values from several independent individual determinations and rounded to two significant places). The IC₅₀ values were determined for racemates and enantiomers from differently generated DMSO stock solutions. Whereas an automatically created DMSO stock solution from the internal substance logistics was used for racemates by means of a standard method, in the case of enantiomers, in each case a freshly produced, manually prepared DMSO stock solution was used for a more precise direct comparison of the enantiomers with one another.

TABLE 3A
Inhibition of MMPs of the mouse

| | MMP-2 | MMP-3 | MMP-7 | MMP-8 | MMP-9 | MMP-12 |
|---|---|---|---|---|---|---|
| Example | IC₅₀ | IC₅₀ | IC₅₀ | IC₅₀ | IC₅₀ | IC₅₀ |
| No. | [nM] | [nM] | [nM] | [nM] | [nM] | [nM] |
| 1 | 33 | 290 | 46 | 71 | 71 | 0.54 |
| 2 | 16.5 | 87 | 22 | 34.5 | 27.5 | 0.13 |
| A-1 | 1.3 | 17 | 15 | 1.1 | 4.9 | <0.61 |
| A-3 | <0.61 | 11.3 | 6.2 | <0.61 | 1.5 | 0.1 |
| B-1 | 610 | 13000 | 1400 | 2300 | 2700 | 32 |
| B-2 | 145 | 6750 | 525 | 1080 | 905 | 18 |

TABLE 3B
Inhibition of MMPs of the rat

| | MMP-2 | MMP-8 | MMP-9 | MMP-12 |
|---|---|---|---|---|
| Example | IC₅₀ | IC₅₀ | IC₅₀ | IC₅₀ |
| No. | [nM] | [nM] | [nM] | [nM] |
| 1 | 33 | 100 | 110 | <1.0 |
| 2 | 16.5 | 38.5 | 46.5 | <0.61 |
| A-1 | 1.3 | 1.4 | 4.6 | <0.61 |
| A-3 | <0.61 | 1.4 | 3.3 | <0.61 |
| B-1 | 610 | 3500 | 3200 | 29.3 |
| B-2 | 145 | 1040 | 1350 | 24.5 |

The compounds according to the invention therefore have a very high inhibitory potency (in the sub-nanomolar range) towards MMP-12 of mouse and rat and at the same time a very high selectivity (generally two orders of magnitude) compared to other MMPs of mouse and rat.

As is evident from the data in Tables 3A/3B, the compounds according to the invention are significantly more potent compared to the relevant comparison compounds as regards MMP-12 (cf. Example 1 to B-1, Example 2 to B-2) or comparably potent (cf. Example 1 to A-1, Example 2 to A-3). Moreover, the compounds according to the invention have a significantly higher selectivity compared to the relevant comparison compounds (as a rule more than one order of magnitude) with regard to other MMPs of mouse and rat.

By virtue of this significantly higher selectivity towards the orthologous MMPs of mouse and rat in combination with the very high potency towards MMP-12, the compounds according to the invention—in contrast to the comparison compounds—are particularly well suited for preclinical investigations in disease models in rodents prior to clinical investigations with human subjects and patients.

As a summarizing assessment of the inhibition data in Tables 1, 2A/2B and 3A/3B, it can therefore be stated that the compounds according to the invention have a very high inhibitory potency both on the human and on the orthologous MMP-12 enzyme of mouse and rat, and moreover exhibit a very high selectivity towards related human or rodent MMPs. The resulting profile in each case of the compounds according to the invention of activity strength and selectivity is always significantly better than that of the listed comparison compounds from the prior art.

### B-3. Animal Model of Pulmonary Emphysema

Elastase-induced pulmonary emphysems in mouse, rat or hamster is a widespread animal model for pulmonary emphysema [The Fas/Fas-ligand pathway does not mediate the apoptosis in elastase-induced emphysema in mice, Sawada et al., Exp. Lung Res. 33, 277-288 (2007)]. The animals receive an orotracheal instillation of porcine pancreas elastase. The treatment of the animals with the test substance starts at the day of the instillation of the porcine pancreas elastase and extends over a period of 3 weeks. At the end of the study, the pulmonary compliance is determined and an alveolar morphometry is carried out.

A further mouse model for pulmonary emphysema is pulmonary emphysema induced by cigarette smoke and an influenza virus infection [Role of ribonuclease L in viral pathogen-associated molecular pattern/influenza virus and cigarette smoke-induced inflammation and remodeling, Zhou et al., J. Immunol. 191, 2637-2646 (2013)]. The animals are exposed to cigarette smoke for several weeks and are then exposed to an influenza virus infection. At the end of the study, a differential cell count in the bronchio-alveolar lavage fluid (BALF) is determined and an alveolar morphometry of the lung is carried out.

### B-4. Animal Model of Silica-Induced Pulmonary Inflammation

An orotracheal administration of silica in mouse, rat or hamster leads to an inflammation in the lung [Involvement of leukotrienes in the pathogenesis of silica-induced pulmonary fibrosis in mice, Shimbori et al., Exp. Lung Res. 36, 292-301 (2010)]. The animals are treated on the day of the instillation of the silica with the test substance. After 24 hours, a bronchio-alveolar lavage is carried out to determine the cell content and the biomarker.

### B-5. Animal Model of Silica-Induced Pulmonary Fibrosis

Silica-induced pulmonary fibrosis in mouse, rat or hamster is a widespread animal model for pulmonary fibrosis [Involvement of leukotrienes in the pathogenesis of silica-induced pulmonary fibrosis in mice, Shimbori et al., Exp. Lung Res. 36, 292-301 (2010)]. The animals receive an orotracheal instillation of silica. The treatment of the animals with the test substance starts on the day of the instillation of the silica or therapeutically a week later and extends over a period of 6 weeks. At the end of the study, a bronchio-alveolar lavage to determine the cell content and the biomarker, and also a histological assessment of pulmonary fibrosis are carried out.

### B-6. Animal Model of ATP-Induced Pulmonary Inflammation

An intratracheal administration of ATP (adenosine triphosphate) on the mouse leads to inflammation in the lung [Acute lung inflammation and ventilator-induced lung injury caused by ATP via the P2Y receptors: An experimental study, Matsuyama et al., Respir. Res. 979 (2008)]. The animals are treated on the day of the instillation of ATP for a period of 24 h with the test substance (by gavage, by addition to feed or drinking water, using an osmotic mini pump, by subcutaneous or intraperitoneal injection or by inhalation). At the end of the experiment, a bronchio-alveolar lavage for determining the cell content and the pro-inflammatory marker is carried out.

### B-7. CYP Inhibition Test

The ability of substances to inhibit the CYP enzymes CYP1A2, CYP2C9, CYP2D6 and CYP3A4 in humans is investigated using pooled human liver microsomes as enzyme source in the presence of standard substrates (see below) which form CYP-specific metabolites. The inhibition effects are investigated at six different concentrations of the test compounds [2.8, 5.6, 8.3, 16.7, 20 (or 25) and 50 μM), compared with the extent of the CYP-specific metabolite formation of the standard substrates in the absence of the test compounds, and the corresponding IC₅₀ values are calculated. A standard inhibitor which specifically inhibits an individual CYP isoform is always co-incubated in order to make the results between different series comparable.

The incubation of phenacetin, diclofenac, tolbutamide, dextromethorphan or midazolam with human liver microsomes in the presence of in each case six different concentrations of a test compound (as potential inhibitor) is carried out on a workstation (Tecan, Genesis, Crailsheim, Germany) Standard incubation mixtures comprise 1.3 mM NADP⁺, 3.3 mM MgCl₂×6 H₂O, 3.3 mM glucose 6-phosphate, glucose 6-phosphate dehydrogenase (0.4 U/ml) and 100 mM phosphate buffer (pH 7.4) in a total volume of 200 μl Test compounds are preferably dissolved in acetonitrile. 96-Well plates are incubated for a defined period of time at 37° C. with pooled human liver microsomes. The reactions are stopped by addition of 100 μl of acetonitrile comprising a suitable internal standard. Precipitated proteins are removed by centrifugation, and the supernatants are combined and analysed by LC-MS/MS.

### B-8. Hepatocyte Assay for Determining the Metabolic Stability

The metabolic stability of test compounds towards hepatocytes is determined by incubating the compounds at low concentrations (preferably below or around 1 μM) and at low cell counts (preferably at 1*10⁶ cells/ml) in order to ensure the greatest possible linear kinetic conditions in the experiment. Seven samples from the incubation solution are removed within a stipulated time frame for the LC-MS analysis in order to determine the half life (i.e. the degradation) of the particular compound. This half life is used to calculate various “Clearance” parameters (CL) and “Fₘₐₓ” values (see below).

The CL and Fₘₐₓ values are a measure of the phase 1 and phase 2 metabolism of the compounds in the hepatocytes. In order to keep the influence of the organic solvent on the enzymes in the incubation mixtures as low as possible, its concentration is generally limited to 1% (acetonitrile) or 0.1% (DMSO).

For all species and races, a hepatocyte cell count in the liver of 1.1*10⁸ cells/g of liver is estimated. CL parameters, the calculation of which is based on half lives which extend considerably beyond the incubation time (usually 90 minutes), can only be regarded as rough guide values.

The calculated parameters and their meaning are:
Fₘₐₓ well-stirred [%] Maximum possible bioavailability following oral application

Calculation: (1−CL_{blood} well-stirred/QH)*100
CL_{blood} well-stirred [L/(h*kg)] Calculated blood clearance (well stirred model)

Calculation: (QH*CL′_{intrinsic}/(QH+CL′_{intrinsic})
CL′_{intrinsic} [ml/(min*kg)] Maximum ability of the liver (of the hepatocytes) to metabolize a compound (assuming that the liver blood flow is not rate-limiting)

Calculation: CL′_{intrinsic, apparent}*species-specific hepatocyte count [1.1*10⁸/g liver]*species-specific liver weight [g/kg]
CL′_{intrinsic, apparent} [ml/(min*mg)] Normalizes the elimination constant by dividing this by the hepatocyte cell count used x (x*10⁶/ml)

Calculation: kₑₗ[1/min]/(cell count [x*10⁶]/incubation volume [ml])

(QH=species-specific liver blood flow).

Table 4 below shows for embodiment example 2 the CL and Fₘₐₓ values from this assay following incubation of the compound with rat hepatocytes (as average value from several independent individual determinations):

TABLE 4
Calculated blood clearance and bioavailability
following incubation with rat hepatocytes

| Example No. | CL_{blood} [L/(h*kg)] | Fₘₐₓ [%] |
|---|---|---|
| 2 | 0.65 | 84.4 |

The specified embodiment example of the present invention thus shows in this model a good pharmacokinetic profile in vitro with a low calculated blood clearance and a high calculated bioavailability.

### B-9. Metabolic Study

To determine the metabolic profile of the inventive compounds, they are incubated with liver microsomes or with primary fresh hepatocytes from various animal species (e.g. rats, dogs), and also of human origin, in order to obtain and to compare information about a very substantially complete hepatic phase I and phase II metabolism, and about the enzymes involved in the metabolism.

The inventive compounds are incubated with a concentration of about 1-10 μM. To this end, stock solutions of the compounds having a concentration of 0.1-1 mM in acetonitrile are prepared, and then pipetted with 1:100 dilution into the incubation mixture. The liver microsomes are incubated at 37° C. in 50 mM potassium phosphate buffer pH 7.4 with and without NADPH-generating system consisting of 1 mM NADP⁺, 10 mM glucose-6-phosphate and 1 unit glucose-6-phosphate dehydrogenase. Primary hepatocytes are incubated in suspension in William's E medium, likewise at 37° C. After an incubation time of 0-4 h, the incubation mixtures are stopped with acetonitrile (final concentration about 30%) and the protein is centrifuged off at about 15 000×g. The samples thus stopped are either analysed directly or stored at −20° C. until analysis.

The analysis is carried out by high-performance liquid chromatography with ultraviolet and mass spectrometry detection (HPLC-UV-MS/MS). To this end, the supernatants of the incubation samples are chromatographed with suitable C18 reversed-phase columns and variable eluent mixtures of acetonitrile and 10 mM aqueous ammonium formate solution or 0.05% aqueous formic acid. The UV chromatograms in conjunction with mass spectrometry data serve for identification, structural elucidation and quantitative estimation of the metabolites, and for quantitative determination of the metabolic reduction of the compounds according to the invention in the incubation mixtures.

### B-10. Pharmacokinetic Investigations In Vivo

The substance to be examined is administered to rats, mice or dogs intravenously as a solution (for example in corresponding plasma with a small addition of DMSO or in a PEG/ethanol/water mixture), and peroral administration is effected as a solution (for example in Solutol/ethanol/water or PEG/ethanol/water mixtures) or as a suspension (e.g. in a water/tylose mixture), in each case via a gavage. After administration of the substance, blood is taken from the animals at fixed times. The blood is heparinized, then plasma is obtained therefrom by centrifugation. The test substance is quantified analytically in the plasma via LC-MS/MS. From the plasma concentration/time plots determined in this way, using an internal standard and with the aid of a validated computer program, the pharmacokinetic parameters, such as AUC (area under the concentration/time curve), Cₘₐₓ (maximum plasma concentration), t_{1/2} (half life), V_{SS} (distribution volume) and CL (clearance), and the absolute and relative bioavailability F and Fᵣₑₗ (i.v./p.o. comparison or comparison of suspension to solution after p.o. administration), are calculated.

Table 5 below shows the pharmacokinetic parameters in rat, mouse and dog for embodiment example 2:

TABLE 5
Pharmacokinetic parameters for embodiment example 2

| | CL_{Plasma} | | | | |
|---|---|---|---|---|---|
| | CLblood | AUCₙₒᵣₘ i.v. | t_{1/2} p.o. | F | Fᵣₑₗ |
| Animal species | [l/h/kg] | [kg*h/L] | [h] | [%] | [%] |
| Rat (Wistar) | 0.011 | 93.6 | 8.4 | 100 | 97 |
| Mouse (C57BL/6) | 0.022 | 44.6 | 5.0 | 84 | n.d. |
| Dog (Beagle) | 0.094 | 10.6 | 14.4 | 100 | n.d. |

[n.d. = not determined].

The specified embodiment example of the present invention thus has in vivo a very low plasma clearance (CL), a long half life (t_{1/2}), a very high exposure (AUC) and a very high bioavailability from solution (F) and also from suspension (Fᵣₑₗ). When viewed overall, the compound according to the invention exhibits a very good pharmacokinetic profile in vivo in the investigated species rat, mouse and dog and thus appears to be suitable to a particular extent for a once-daily, oral administration in a low dosage to humans.

### C. EMBODIMENT EXAMPLES OF PHARMACEUTICAL COMPOSITIONS

The compounds according to the invention can be converted to pharmaceutical formulations as follows:

### Tablet:

### Composition:

100 mg of the compound according to the invention, 50 mg of lactose (monohydrate), 50 mg of corn starch (native), 10 mg of polyvinylpyrrolidone (PVP 25)→(BASF, Ludwigshafen, Germany) and 2 mg of magnesium stearate.

Tablet weight 212 mg. Diameter 8 mm, radius of curvature 12 mm

### Preparation:

The mixture of inventive compound, lactose and starch is granulated with a 5% solution (w/w) of the PVP in water. The granules are dried and then mixed with the magnesium stearate for 5 minutes. This mixture is compressed in a conventional tablet press (see above for format of the tablet). The guide value used for the pressing is a pressing force of 15 kN.

Suspension which can be Administered Orally:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

### Preparation:

The Rhodigel is suspended in ethanol; the compound according to the invention is added to the suspension. The water is added while stirring. The mixture is stirred for about 6 h before swelling of the Rhodigel is complete.

### Solution for Oral Administration:

### Composition:

500 mg of the compound according to the invention, 2.5 g of polysorbate and 97 g of polyethylene glycol 400. 20 g of oral solution correspond to a single dose of 100 mg of the compound according to the invention.

### Preparation:

The compound according to the invention is suspended in the mixture of polyethylene glycol and polysorbate with stirring. The stirring operation is continued until dissolution of the compound according to the invention is complete.

i.v. Solution:

The compound according to the invention is dissolved in a concentration below the saturation solubility in a physiologically acceptable solvent (e.g. isotonic saline solution, glucose solution 5% and/or PEG 400 solution 30%). The solution is subjected to sterile filtration and dispensed into sterile and pyrogen-free injection vessels.

1. (1S,2S,5R)-2-[4-(Benzyloxy)benzoyl]-5-[(4-oxo-1,2,3-benzotriazin-3(4H)-yl)methyl]cyclopentanecarboxylic acid of the formula (I-A) or (1R,2R,5S)-2-[4-(benzyloxy)benzoyl]-5-[(4-oxo-1,2,3-benzotriazin-3(4H)-yl)methyl]cyclopentanecarboxylic acid of the formula (I-B) 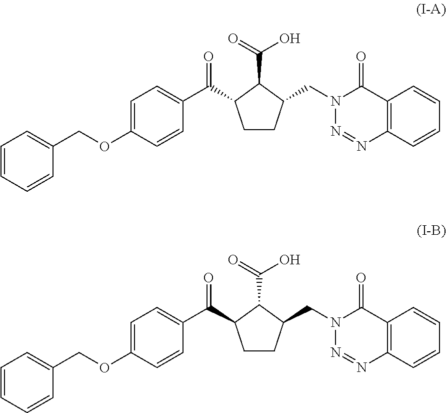 [SMILES: O=C(O)[C@H]1[C@H](Cn2nnc3ccccc3c2=O)CC[C@@H]1C(=O)c1ccc(OCc2ccccc2)cc1 ; O=C(O)[C@@H]1[C@@H](Cn2nnc3ccccc3c2=O)CC[C@H]1C(=O)c1ccc(OCc2ccccc2)cc1]
or a mixture of these compounds or a salt, solvate or solvate of a salt of these compounds or their mixture.

2. Mixture of the compounds of the formula (I-A) and (I-B) according claim 1, wherein the compounds of the formula (I-A) and (I-B) are present as racemic mixture, or a salt, solvate or solvate of a salt of this racemic mixture.

3. Compounds according to claim 1 of the formula (I-A) 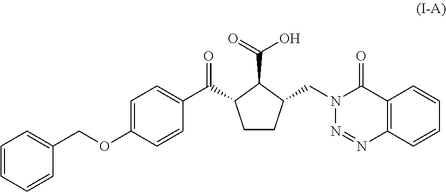 [SMILES: O=C(O)[C@H]1[C@H](Cn2nnc3ccccc3c2=O)CC[C@@H]1C(=O)c1ccc(OCc2ccccc2)cc1]
in enantiomerically pure form or a salt, solvate or solvate of a salt thereof.

4. Process for preparing a compound or a mixture of compounds, as defined in claim 1, wherein exo-2-(trimethylsilyl)ethyl 2-oxobicyclo[2.2.1]heptane-7-carboxylate of the formula (II) 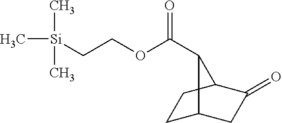 [SMILES: C[Si](C)(C)CCOC(=O)C1C2CCC1C(=O)C2]
is reacted with a phenyl Grignard compound of the formula (III) 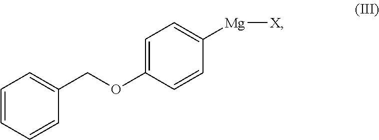
in which X is chlorine, bromine or iodine,
to give the adduct of the formula (IV) 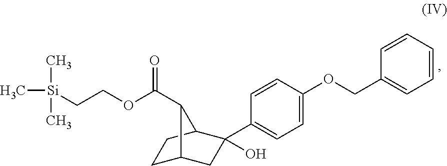 [SMILES: C[Si](C)(C)CCOC(=O)C1C2CCC1C(O)(c1ccc(OCc3ccccc3)cc1)C2]
then the hydroxy group is eliminated via the mesylate produced in situ of the formula (V) 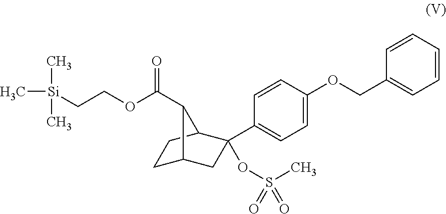 [SMILES: C[Si](C)(C)CCOC(=O)C1C2CCC1C(OS(C)(=O)=O)(c1ccc(OCc3ccccc3)cc1)C2]
to give the olefin of the formula (VI) 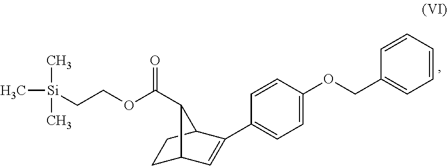 [SMILES: C[Si](C)(C)CCOC(=O)C1C2C=C(c3ccc(OCc4ccccc4)cc3)C1CC2]
then oxidation is carried out with N-methylmorpholine N-oxide together with osmium tetroxide as catalyst to give the cis-1,2-diol of the formula (VII) 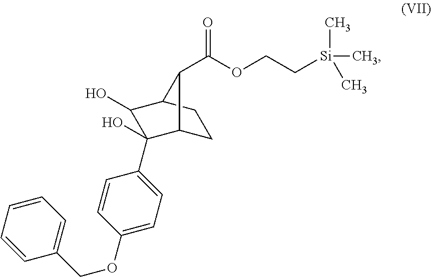 [SMILES: C[Si](C)(C)CCOC(=O)C1C2CCC1C(O)(c1ccc(OCc3ccccc3)cc1)C2O]
then this bicyclic diol is cleaved with the help of lead tetraacetate or sodium periodate to give the racemic mixture of the 2-benzoyl-5-formylcyclopentanecarboxylic acid esters (VIII-A) and (VIII-B) 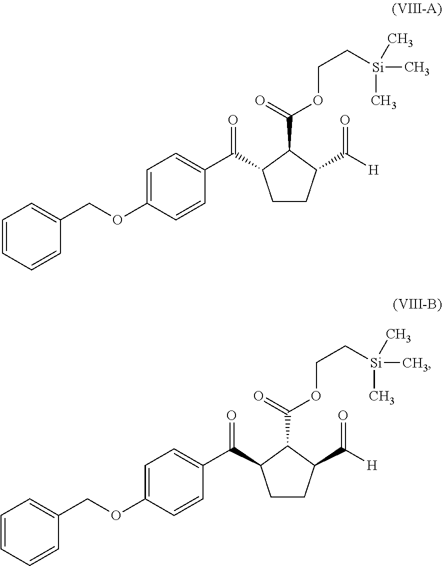 [SMILES: C[Si](C)(C)CCOC(=O)[C@H]1[C@H](C=O)CC[C@@H]1C(=O)c1ccc(OCc2ccccc2)cc1 ; C[Si](C)(C)CCOC(=O)[C@@H]1[C@@H](C=O)CC[C@H]1C(=O)c1ccc(OCc2ccccc2)cc1]
this mixture is reduced with sodium borohydride to give the racemic mixture of the hydroxymethyl compounds (IX-A) and (IX-B) 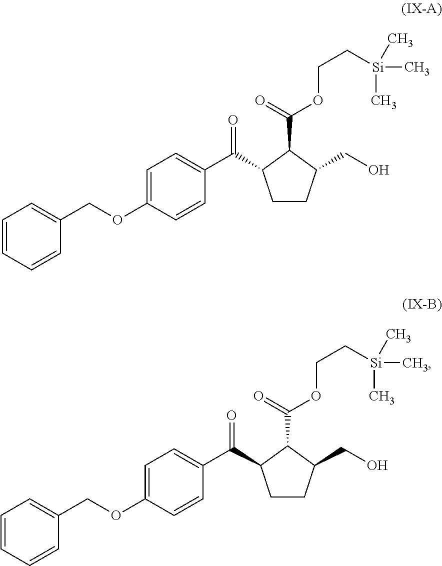 [SMILES: C[Si](C)(C)CCOC(=O)[C@H]1[C@H](CO)CC[C@@H]1C(=O)c1ccc(OCc2ccccc2)cc1 ; C[Si](C)(C)CCOC(=O)[C@@H]1[C@@H](CO)CC[C@H]1C(=O)c1ccc(OCc2ccccc2)cc1]
then reaction is carried out with 1,2,3-benzotriazin-4(3H)-one of the formula (X) 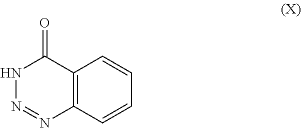 [SMILES: O=c1[nH]nnc2ccccc12]
in the presence of an alkyl- or arylphosphane and an azodicarboxylate to give the racemic mixture of the benzotriazinone derivatives (XI-A) and (XI-B) 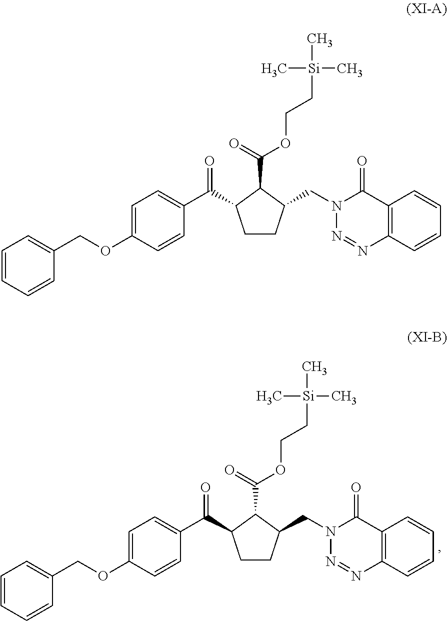 [SMILES: C[Si](C)(C)CCOC(=O)[C@H]1[C@H](Cn2nnc3ccccc3c2=O)CC[C@@H]1C(=O)c1ccc(OCc2ccccc2)cc1 ; C[Si](C)(C)CCOC(=O)[C@@H]1[C@@H](Cn2nnc3ccccc3c2=O)CC[C@H]1C(=O)c1ccc(OCc2ccccc2)cc1]
and finally the 2-(trimethylsilyl)ethyl ester group is cleaved off with the help of an acid or of a fluoride reagent to give the racemic mixture of the cyclopentanecarboxylic acids according to the invention (I-A) and (I-B) 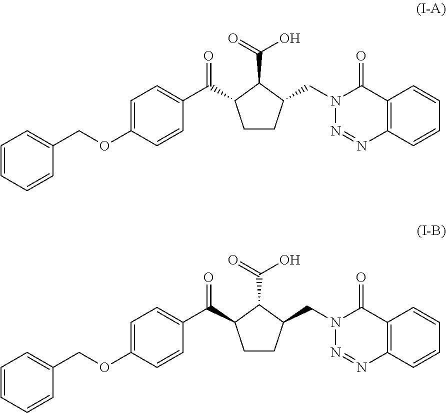 [SMILES: O=C(O)[C@H]1[C@H](Cn2nnc3ccccc3c2=O)CC[C@@H]1C(=O)c1ccc(OCc2ccccc2)cc1 ; O=C(O)[C@@H]1[C@@H](Cn2nnc3ccccc3c2=O)CC[C@H]1C(=O)c1ccc(OCc2ccccc2)cc1]
and optionally the resulting mixture of the compounds (I-A) and (I-B) is separated into the enantiomerically pure compounds and/or converted with the corresponding (i) solvents and/or (ii) bases to the solvates, salts and/or solvates of the salts.

5. Compound or mixture of compounds, as defined in claim 1, for the treatment and/or prevention of diseases.

6. Compound or mixture of compounds, as defined in claim 1, for use in a method for the treatment and/or prevention of chronic obstructive pulmonary disease (COPD), pulmonary emphysema, chronic bronchitis, pulmonary hypertension in the COPD (PH-COPD), bronchiectasis, asthma, interstitial pulmonary disorders, idiopathic pulmonary fibrosis (IPF) and pulmonary sarcoidosis, of arteriosclerosis, carotid arteriosclerosis, viral myocarditis, cardiomyopathy and aneurysms, including their consequential diseases such as stroke, myocardial infarction and peripheral arterial occlusive disease, and also of chronic kidney diseases and Alport's syndrome.

7. Use of a compound or mixture of compounds, as defined in claim 1, for producing a medicament for the treatment and/or prevention of chronic obstructive pulmonary disease (COPD), pulmonary emphysema, chronic bronchitis, pulmonary hypertension in the COPD (PH-COPD), bronchiectasis, asthma, interstitial pulmonary disorders, idiopathic pulmonary fibrosis (IPF) and pulmonary sarcoidosis, of arteriosclerosis, carotid arteriosclerosis, viral myocarditis, cardiomyopathy and aneurysms, including their consequential diseases such as stroke, myocardial infarction and peripheral arterial occlusive disease, and also of chronic kidney diseases and Alport's syndrome.

8. Medicament comprising a compound or a mixture of compounds, as defined in claim 1, in combination with one or more inert non-toxic, pharmaceutically suitable auxiliaries.

9. Medicament comprising a compound or a mixture of compounds, as defined in claim 1, in combination with one or more further active ingredients selected from the group consisting of corticosteroids, beta-adrenergic receptor agonists, antimuscarinic substances, PDE 4 inhibitors, PDE 5 inhibitors, sGC activators, sGC stimulators, HNE inhibitors, prostacyclin analogues, endothelin antagonists, statins, antifibrotic agents, antiinflammatory agents, immunomodulating agents, immunosuppressive agents and cytotoxic agents.

10. Medicament according to claim 8, for the treatment and/or prevention of chronic obstructive pulmonary disease (COPD), pulmonary emphysema, chronic bronchitis, pulmonary hypertension in the COPD (PH-COPD), bronchiectasis, asthma, interstitial pulmonary disorders, idiopathic pulmonary fibrosis (IPF) and pulmonary sarcoidosis, of arteriosclerosis, carotid arteriosclerosis, viral myocarditis, cardiomyopathy and aneurysms, including their consequential diseases such as stroke, myocardial infarction and peripheral arterial occlusive disease, and also of chronic kidney diseases and Alport's syndrome.

11. Method for the treatment and/or prevention of chronic obstructive pulmonary disease (COPD), pulmonary emphysema, chronic bronchitis, pulmonary hypertension in the COPD (PH-COPD), bronchiectasis, asthma, interstitial pulmonary disorders, idiopathic pulmonary fibrosis (IPF) and pulmonary sarcoidosis, of arteriosclerosis, carotid arteriosclerosis, viral myocarditis, cardiomyopathy and aneurysms, including their consequential diseases such as stroke, myocardial infarction and peripheral arterial occlusive disease, and also of chronic kidney diseases and Alport's syndrome in humans and animals by administering an effective amount of a compound or of a mixture of compounds, as defined in claim 1, or of a medicament comprising the compound or the mixture auxiliaries.
